SEQUENCE LISTING (178 sequences)

SEQ ID NO: 1 - protein, 1046 aa, Artificial Sequence
  Amino acid sequence of the N-terminal fragment of SpRY(D10A) protein
DKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFK

SEQ ID NO: 2 - DNA, 3138 bp, Artificial Sequence
  Nucleotide sequence encoding the N-terminal fragment of SpRY(D10A) protein
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaag

SEQ ID NO: 3 - protein, 166 aa, Artificial Sequence
  Amino acid sequence of the TadA8e fragment
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 4 - DNA, 498 bp, Artificial Sequence
  The nucleotide sequence encoding the TadA8e fragment
tctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaac

SEQ ID NO: 5 - protein, 305 aa, Artificial Sequence
  Amino acid sequence of the C-terminal fragment of SpRY(D10A) protein
ETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 6 - DNA, 915 bp, Artificial Sequence
  Nucleotide sequence encoding the C-terminal fragment of SpRY(D10A) protein
gagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgac

SEQ ID NO: 7 - protein, 32 aa, Artificial Sequence
  Sequence of linker peptide contained in the fusion protein CE-8e-SpRY
SGGSSGGSSGSETPGTSESATPESSGGSSGGS

SEQ ID NO: 8 - DNA, 96 bp, Artificial Sequence
  Nucleotide sequence encoding the linker peptide in CE-8e-SpRY
tctggaggatctagcggtggttcctctggaagcgagacaccaggcacaagcgagtccgccacaccagagagctccggcggctcctccggaggatcc

SEQ ID NO: 9 - protein, 18 aa, Artificial Sequence
  Amino acid sequence of the nuclear localization signal at both ends of fusion protein CE-8e-SpRY (bpNLS)
KRTADGSEFESPKKKRKV

SEQ ID NO: 10 - DNA, 54 bp, Artificial Sequence
  Nucleotide sequence of the nuclear localization signal at both ends of the fusion protein CE-8e-SpRY (bpNLS)
aaacggacagccgacggaagcgagttcgagtcaccaaagaagaagcggaaagtc

SEQ ID NO: 11 - protein, 7 aa, Artificial Sequence
  Amino acid sequence of the nuclear localization signal fragment(SV40NLS)
PKKKRKV

SEQ ID NO: 12 - DNA, 21 bp, Artificial Sequence
  Nucleotide sequence of nuclear localization signal (SV40NLS)
ccaaagaagaagcggaaagtc

SEQ ID NO: 13 - protein, 1581 aa, Artificial Sequence)
  Amino acid sequence of CE-8e-SpRY fusion protein
DKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 14 - DNA, 4743 bp, Artificial Sequence
  Nucleotide sequence of CE-8e-SpRY fusion protein
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagtctggaggatctagcggtggttcctctggaagcgagacaccaggcacaagcgagtccgccacaccagagagctccggcggctcctccggaggatcctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgac

SEQ ID NO: 15 - DNA, 498 bp, Artificial Sequence
  Nucleotide sequence of TadA8e V106W
tctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggctggaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaac

SEQ ID NO: 16 - DNA, 4101 bp, Artificial Sequence
  Nucleotide sequence of SpRY(D10A)
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgac

SEQ ID NO: 17 - DNA, 4101 bp, Artificial Sequence
  Nucleotide sequence of SpRY D10A-HF
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgagagaacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccgccttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcgcgctgagccggaagctgatcaacggcatccgggacaagcagtccggcaagacaatcctggatttcctgaagtccgacggcttcgccaacagaaacttcatggcgctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccgggcgatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcagacccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccagactgggagcccctagagccttcaagtactttgacaccaccatcgaccccaagcagtacagaagcaccaaagaggtgctggacgccaccctgatccaccagagcatcaccggcctgtacgagacacggatcgacctgtctcagctgggaggtgac

SEQ ID NO: 18 - DNA, 20 bp, Artificial Sequence
  sgRNA1
cctgcttgcaagtgtcaacc

SEQ ID NO: 19 - DNA, 20 bp, Artificial Sequence
  sgRNA2
tacttgcgacagttccttcc

SEQ ID NO: 20 - DNA, 20 bp, Artificial Sequence
  sgRNA3
catttccctctacgctcgct

SEQ ID NO: 21 - DNA, 20 bp, Artificial Sequence
  sgRNA4
gtgaaacaccacgcccggcc

SEQ ID NO: 22 - DNA, 20 bp, Artificial Sequence
  sgRNA5
cctcttctggaaaggggtac

SEQ ID NO: 23 - DNA, 20 bp, Artificial Sequence
  sgRNA6
gacagttccttccaattcca

SEQ ID NO: 24 - DNA, 20 bp, Artificial Sequence
  sgRNA7
aacaacagtacttgcgacag

SEQ ID NO: 25 - DNA, 20 bp, Artificial Sequence
  sgRNA8
gcagcagcctggaaaagtac

SEQ ID NO: 26 - DNA, 20 bp, Artificial Sequence
  sgRNA9
gatgcagcgaatgtgaaatc

SEQ ID NO: 27 - DNA, 20 bp, Artificial Sequence
  sgRNA10
tcatcttaggccttcaagga

SEQ ID NO: 28 - DNA, 20 bp, Artificial Sequence
  sgRNA11
gagatgcagcgaatgtgaaa

SEQ ID NO: 29 - DNA, 20 bp, Artificial Sequence
  sgRNA12
gagcagggaagaaggaatca

SEQ ID NO: 30 - DNA, 20 bp, Artificial Sequence
  sgRNA13
gaaaagtacttggggaccaa

SEQ ID NO: 31 - DNA, 20 bp, Artificial Sequence
  sgRNA14
ttggaactcctgcttgcaag

SEQ ID NO: 32 - DNA, 20 bp, Artificial Sequence
  sgRNA15
aaggccaagcttgcctgccc

SEQ ID NO: 33 - DNA, 20 bp, Artificial Sequence
  sgRNA16
aaagctgcatccaccccccg

SEQ ID NO: 34 - DNA, 20 bp, Artificial Sequence
  sgRNA17
ctgctagacgacagcgcagg

SEQ ID NO: 35 - DNA, 20 bp, Artificial Sequence
  sgRNA18
gtaacctcccttgaaagggg

SEQ ID NO: 36 - DNA, 20 bp, Artificial Sequence
  sgRNA19
ggagcatctgagtccagggg

SEQ ID NO: 37 - DNA, 20 bp, Artificial Sequence
  sgRNA20
ctagacgacagcgcaggcaa

SEQ ID NO: 38 - DNA, 20 bp, Artificial Sequence
  sgRNA21
ccgaatgcaaaggttctctg

SEQ ID NO: 39 - DNA, 20 bp, Artificial Sequence
  sgRNA22
ccgtccaaagtcaaattgct

SEQ ID NO: 40 - DNA, 20 bp, Artificial Sequence
  sgRNA23
tggcacatagatagtaagtg

SEQ ID NO: 41 - DNA, 20 bp, Artificial Sequence
  sgRNA24
tggccgaatgcaaaggttct

SEQ ID NO: 42 - DNA, 20 bp, Artificial Sequence
  sgRNA26
tagactaattatataagcct

SEQ ID NO: 43 - DNA, 20 bp, Artificial Sequence
  sgRNA26
tggctaagcatcaggagttt

SEQ ID NO: 44 - DNA, 20 bp, Artificial Sequence
  sgRNA27
gagttccacccgctgtgcgt

SEQ ID NO: 45 - DNA, 20 bp, Artificial Sequence
  sgRNA28
atgcaaaggttctctgctag

SEQ ID NO: 46 - DNA, 20 bp, Artificial Sequence
  sgRNA29
gaatgcaaaggttctctgct

SEQ ID NO: 47 - DNA, 20 bp, Artificial Sequence
  sgRNA30
tcggccaatggggcacaagg

SEQ ID NO: 48 - DNA, 20 bp, Artificial Sequence
  sgRNA31
gttacaccaaagggctagaa

SEQ ID NO: 49 - DNA, 20 bp, Artificial Sequence
  sgRNA32
ggttacaccaaagggctaga

SEQ ID NO: 50 - DNA, 20 bp, Artificial Sequence
  sgRNA33
taccaacaatagaggcccat

SEQ ID NO: 51 - DNA, 20 bp, Artificial Sequence
  sgRNA34
ctgtcaaactgtgcgtatga

SEQ ID NO: 52 - DNA, 20 bp, Artificial Sequence
  sgRNA35
accaacaatagaggcccatt

SEQ ID NO: 53 - DNA, 20 bp, Artificial Sequence
  sgRNA36
gtttacataaaagatcttca

SEQ ID NO: 54 - DNA, 20 bp, Artificial Sequence
  sgRNA37
ctaccaacaatagaggccca

SEQ ID NO: 55 - DNA, 20 bp, Artificial Sequence
  sgRNA38
acatcatcagatattctgca

SEQ ID NO: 56 - DNA, 20 bp, Artificial Sequence
  sgRNA39
gctgcaaacaagtgcagaat

SEQ ID NO: 57 - DNA, 20 bp, Artificial Sequence
  sgRNA40
gtctcagaggatgtgttgtt

SEQ ID NO: 58 - DNA, 20 bp, Artificial Sequence
  sgRNA41
ctgcagcccaagcctcagtg

SEQ ID NO: 59 - DNA, 20 bp, Artificial Sequence
  sgRNA42
tcctaaaccagtgtcaggga

SEQ ID NO: 60 - DNA, 20 bp, Artificial Sequence
  sgRNA43
aaagatcttcacaggctacc

SEQ ID NO: 61 - DNA, 20 bp, Artificial Sequence
  sgRNA44
ccaacaatagaggcccatta

SEQ ID NO: 62 - DNA, 20 bp, Artificial Sequence
  sgRNA45
aggtcctaaaccagtgtcag

SEQ ID NO: 63 - DNA, 20 bp, Artificial Sequence
  sgRNA46
aatacaaatagttaagaaca

SEQ ID NO: 64 - DNA, 20 bp, Artificial Sequence
  sgRNA47
ctggaacacaaagcatagac

SEQ ID NO: 65 - DNA, 20 bp, Artificial Sequence
  sgRNA48
gaacacaaagcatagactgc

SEQ ID NO: 66 - DNA, 20 bp, Artificial Sequence
  The sgRNA sequence used for testing RNA off- target
ctggaacacaaagcatagac

SEQ ID NO: 67 - protein, 1803 aa, Artificial Sequence
  Amino acid sequence of ABEmax-SpRY fusion protein
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSKRTADGSEFEPKKKRKV

SEQ ID NO: 68 - protein, 1605 aa, Artificial Sequence
  Amino acid sequence of 8e-SpRY fusion protein
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSKRTADGSEFEPKKKRKV

SEQ ID NO: 69 - protein, 1605 aa, Artificial Sequence
  Amino acid sequence of V106W-SpRY fusion protein
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSKRTADGSEFEPKKKRKV

SEQ ID NO: 70 - protein, 1605 aa, Artificial Sequence
  Amino acid sequence of 8e-SpRY-HF fusion protein
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSKRTADGSEFEPKKKRKV

SEQ ID NO: 71 - protein, 1605 aa, Artificial Sequence
  Amino acid sequence of V106W-SpRY-HF fusion protein
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSKRTADGSEFEPKKKRKV

SEQ ID NO: 72 - DNA, 20 bp, Artificial Sequence
  Upstream primer for sg1 identification
gagtagccaaagaccatcag

SEQ ID NO: 73 - DNA, 20 bp, Artificial Sequence
  Downstream primer for sg1 identification
cttccataacaaaaggaagc

SEQ ID NO: 74 - DNA, 20 bp, Artificial Sequence
  Upstream primer for sg2 identification
tgtttttgtggaacatggtg

SEQ ID NO: 75 - DNA, 20 bp, Artificial Sequence
  Downstream primer for sg2 identification
gaatcaggcaagaaataagc

SEQ ID NO: 76 - DNA, 20 bp, Artificial Sequence
  Upstream primer for sg3 identification
gacttgtggagatgcagcga

SEQ ID NO: 77 - DNA, 19 bp, Artificial Sequence
  Upstream primer for sg4 identification
acagggttttgccgtgttg

SEQ ID NO: 78 - DNA, 18 bp, Artificial Sequence
  Downstream primer for sg4 identification
gcccgttaggactgcttg

SEQ ID NO: 79 - DNA, 20 bp, Artificial Sequence
  Upstream primer for sg5 identification
cttgtggagatgcagcgaat

SEQ ID NO: 80 - DNA, 20 bp, Artificial Sequence
  Downstream primer for sg5 identification
aagcactcaccatgttccac

SEQ ID NO: 81 - DNA, 20 bp, Artificial Sequence
  Upstream primer for sg6 identification
gtggaacatggtgagtgctt

SEQ ID NO: 82 - DNA, 20 bp, Artificial Sequence
  Downstream primer for sg6 identification
tagcagaggaatcaggcaag

SEQ ID NO: 83 - DNA, 20 bp, Artificial Sequence
  Upstream primer for sg7 identification
acactgcaatgtttttgtgg

SEQ ID NO: 84 - DNA, 20 bp, Artificial Sequence
  Downstream primer for sg7 identification
aggcaagaaataagcaactc

SEQ ID NO: 85 - DNA, 20 bp, Artificial Sequence
  Upstream primer for sg8 identification
gtggagatgcagcgaatgtg

SEQ ID NO: 86 - DNA, 20 bp, Artificial Sequence
  Downstream primer for sg8 identification
gaaaagcactcaccatgttc

SEQ ID NO: 87 - DNA, 20 bp, Artificial Sequence
  Upstream primer for sg9 identification
gcttccttttgttatggaag

SEQ ID NO: 88 - DNA, 20 bp, Artificial Sequence
  Downstream primer for sg9 identification
gatgattccttcttccctgc

SEQ ID NO: 89 - DNA, 20 bp, Artificial Sequence
  Upstream primer for sg10 identification
atgggggtccacactgcaat

SEQ ID NO: 90 - DNA, 20 bp, Artificial Sequence
  Downstream primer for sg10 identification
ccgaagtcacgaagtaactt

SEQ ID NO: 91 - DNA, 20 bp, Artificial Sequence
  Upstream primer for sg11 identification
gtgcttccttttgttatgga

SEQ ID NO: 92 - DNA, 20 bp, Artificial Sequence
  Downstream primer for sg11 identification
gattccttcttccctgctcc

SEQ ID NO: 93 - DNA, 20 bp, Artificial Sequence
  Upstream primer for sg12 identification
ctggaaaggggtacctatta

SEQ ID NO: 94 - DNA, 20 bp, Artificial Sequence
  Downstream primer for sg12 identification
ttggaaggaactgtcgcaag

SEQ ID NO: 95 - DNA, 20 bp, Artificial Sequence
  Upstream primer for sg13 identification
agatcttgaaccctcttctg

SEQ ID NO: 96 - DNA, 20 bp, Artificial Sequence
  Downstream primer for sg13 identification
gttcaaagttccttgaaggc

SEQ ID NO: 97 - DNA, 20 bp, Artificial Sequence
  Upstream primer for sg14 identification
gaggagtagccaaagaccat

SEQ ID NO: 98 - DNA, 20 bp, Artificial Sequence
  Downstream primer for sg14 identification
tccataacaaaaggaagcac

SEQ ID NO: 99 - DNA, 20 bp, Artificial Sequence
  Upstream primer for sg15 identification
tgaaccctcttctggaaagg

SEQ ID NO: 100 - DNA, 20 bp, Artificial Sequence
  Downstream primer for sg15 identification
ctgttgttcaaagttccttg

SEQ ID NO: 101 - DNA, 20 bp, Artificial Sequence
  Upstream primer for sg16 identification
ggcgtggcagctgatatccg

SEQ ID NO: 102 - DNA, 23 bp, Artificial Sequence
  Downstream primer for sg16 identification
gtgtttcacaaaacaggggtggc

SEQ ID NO: 103 - DNA, 20 bp, Artificial Sequence
  Downstream primer for sg3 identification
cactcaccatgttccacaaa

SEQ ID NO: 104 - DNA, 20 bp, Artificial Sequence
  Upstream primer for sg17 identification
gagcatctgagtccagggga

SEQ ID NO: 105 - DNA, 20 bp, Artificial Sequence
  Downstream primer for sg17 identification
gacagggtagacggcagttc

SEQ ID NO: 106 - DNA, 20 bp, Artificial Sequence
  Upstream primer for sg18 identification
cactccttgtgccccattgg

SEQ ID NO: 107 - DNA, 20 bp, Artificial Sequence
  Downstream primer for sg18 identification
gttgtagtagtcgcgactct

SEQ ID NO: 108 - DNA, 20 bp, Artificial Sequence
  Upstream primer for sg19 identification
cagtatccgaaggcagcagc

SEQ ID NO: 109 - DNA, 20 bp, Artificial Sequence
  D
cattcggccaatggggcaca

SEQ ID NO: 110 - DNA, 20 bp, Artificial Sequence
  Upstream primer for sg20 identification
tacttagggggcacttcgac

SEQ ID NO: 111 - DNA, 20 bp, Artificial Sequence
  Downstream primer for sg20 identification
aaggggaatactcagcagta

SEQ ID NO: 112 - DNA, 20 bp, Artificial Sequence
  Upstream primer for sg21 identification
cgttggagcatctgagtcca

SEQ ID NO: 113 - DNA, 20 bp, Artificial Sequence
  Downstream primer for sg21 identification
gacggcagttcaagtgtccc

SEQ ID NO: 114 - DNA, 18 bp, Artificial Sequence
  Downstream primer for sg22 identification
tgttcacgggctgcgctg

SEQ ID NO: 115 - DNA, 19 bp, Artificial Sequence
  Downstream primer for sg22 identification
ggtggatgcagctttgcgg

SEQ ID NO: 116 - DNA, 23 bp, Artificial Sequence
  Upstream primer for sg23 identification
ttaatgagtggcatcttggcata

SEQ ID NO: 117 - DNA, 24 bp, Artificial Sequence
  Downstream primer for sg23 identification
ttgcaccaaaatggttagtaaaac

SEQ ID NO: 118 - DNA, 20 bp, Artificial Sequence
  Upstream primer for sg24 identification
tggagcatctgagtccaggg

SEQ ID NO: 119 - DNA, 20 bp, Artificial Sequence
  Downstream primer for sg24 identification
ggtagacggcagttcaagtg

SEQ ID NO: 120 - DNA, 23 bp, Artificial Sequence
  Upstream primer for sg25 identification
ctttgagatggaatggagttttg

SEQ ID NO: 121 - DNA, 22 bp, Artificial Sequence
  Downstream primer for sg25 identification
agaaacatgaactgtgagatgc

SEQ ID NO: 122 - DNA, 18 bp, Artificial Sequence
  Upstream primer for sg26 identification
gacacaagcagggacacg

SEQ ID NO: 123 - DNA, 21 bp, Artificial Sequence
  Downstream primer for sg26 identification
ctactctggaggcgaaaaagg

SEQ ID NO: 124 - DNA, 20 bp, Artificial Sequence
  Upstream primer for sg27 identification
cgccaaggagttgtgtaagg

SEQ ID NO: 125 - DNA, 20 bp, Artificial Sequence
  Downstream primer for sg27 identification
tctcgccttctagccctttg

SEQ ID NO: 126 - DNA, 20 bp, Artificial Sequence
  Upstream primer for sg28 identification
gaacagcttcggggggattg

SEQ ID NO: 127 - DNA, 20 bp, Artificial Sequence
  Downstream primer for sg28 identification
cttgtagagagacagggtag

SEQ ID NO: 128 - DNA, 20 bp, Artificial Sequence
  Upstream primer for sg29 identification
gggattgcatgtacgcccca

SEQ ID NO: 129 - DNA, 20 bp, Artificial Sequence
  Downstream primer for sg29 identification
ccagtgctccggacttgtag

SEQ ID NO: 130 - DNA, 20 bp, Artificial Sequence
  Upstream primer for sg30 identification
ggcacttcgaccatttctga

SEQ ID NO: 131 - DNA, 20 bp, Artificial Sequence
  Downstream primer for sg30 identification
tatcttcagtgctcttgcct

SEQ ID NO: 132 - DNA, 20 bp, Artificial Sequence
  Upstream primer for sg31 identification
ccgaatgcaaaggttctctg

SEQ ID NO: 133 - DNA, 20 bp, Artificial Sequence
  Downstream primer for sg31 identification
ccagagccagtggaaagttg

SEQ ID NO: 134 - DNA, 20 bp, Artificial Sequence
  Upstream primer for sg32 identification
gtacgccccacttttgggag

SEQ ID NO: 135 - DNA, 20 bp, Artificial Sequence
  Downstream primer for sg32 identification
gtccagtgctccggacttgt

SEQ ID NO: 136 - DNA, 21 bp, Artificial Sequence
  Upstream primer for sg32 identification
cttcacaggctaccccctaag

SEQ ID NO: 137 - DNA, 20 bp, Artificial Sequence
  Downstream primer for sg32 identification
gaaaattaccacatgctgtc

SEQ ID NO: 138 - DNA, 20 bp, Artificial Sequence
  Upstream primer for sg34 identification
atgtggtaattttccagccc

SEQ ID NO: 139 - DNA, 20 bp, Artificial Sequence
  Downstream primer for sg34 identification
caagcacatgacagttaagg

SEQ ID NO: 140 - DNA, 20 bp, Artificial Sequence
  Upstream primer for sg35 identification
gatcttcacaggctaccccc

SEQ ID NO: 141 - DNA, 20 bp, Artificial Sequence
  Downstream primer for sg35 identification
ccacatgctgtcacagttag

SEQ ID NO: 142 - DNA, 20 bp, Artificial Sequence
  Upstream primer for sg36 identification
gcagtgtttagaaggagact

SEQ ID NO: 143 - DNA, 20 bp, Artificial Sequence
  Downstream primer for sg36 identification
gttggtagaatggcagtgca

SEQ ID NO: 144 - DNA, 20 bp, Artificial Sequence
  Upstream primer for sg37 identification
cacaggctaccccctaagtc

SEQ ID NO: 145 - DNA, 20 bp, Artificial Sequence
  Downstream primer for sg37 identification
ggctggaaaattaccacatg

SEQ ID NO: 146 - DNA, 20 bp, Artificial Sequence
  Upstream primer for sg38 identification
cagcatgtggtaattttcca

SEQ ID NO: 147 - DNA, 20 bp, Artificial Sequence
  Downstream primer for sg38 identification
cacatgacagttaaggtttg

SEQ ID NO: 148 - DNA, 20 bp, Artificial Sequence
  Upstream primer for sg39 identification
caagacctggctgagctaac

SEQ ID NO: 149 - DNA, 20 bp, Artificial Sequence
  Downstream primer for sg39 identification
tttagtctttcaagcaggtg

SEQ ID NO: 150 - DNA, 22 bp, Artificial Sequence
  Upstream primer for sg40 identification
aattgtttgaacctgggagttg

SEQ ID NO: 151 - DNA, 24 bp, Artificial Sequence
  Downstream primer for sg40 identification
gttgatagcctcacagaaagtaac

SEQ ID NO: 152 - DNA, 20 bp, Artificial Sequence
  Upstream primer for sg41 identification
cctaaaccagtgtcagggag

SEQ ID NO: 153 - DNA, 20 bp, Artificial Sequence
  Downstream primer for sg41 identification
tgctttgtgttccagtttcc

SEQ ID NO: 154 - DNA, 20 bp, Artificial Sequence
  Upstream primer for sg42 identification
agaaggagacttgtgcacat

SEQ ID NO: 155 - DNA, 20 bp, Artificial Sequence
  Downstream primer for sg42 identification
attgttggtagaatggcagt

SEQ ID NO: 156 - DNA, 20 bp, Artificial Sequence
  Upstream primer for sg43 identification
gtttagaaggagacttgtgc

SEQ ID NO: 157 - DNA, 20 bp, Artificial Sequence
  Downstream primer for sg43 identification
ggtagaatggcagtgcaata

SEQ ID NO: 158 - DNA, 20 bp, Artificial Sequence
  Upstream primer for sg44 identification
aaagatcttcacaggctacc

SEQ ID NO: 159 - DNA, 20 bp, Artificial Sequence
  Downstream primer for sg44 identification
ccagcgggctggaaaattac

SEQ ID NO: 160 - DNA, 20 bp, Artificial Sequence
  Upstream primer for sg45 identification
ggagacttgtgcacattcta

SEQ ID NO: 161 - DNA, 20 bp, Artificial Sequence
  Downstream primer for sg45 identification
tctattgttggtagaatggc

SEQ ID NO: 162 - DNA, 20 bp, Artificial Sequence
  Upstream primer for sg46 identification
ggccctgtaaaggaaactgg

SEQ ID NO: 163 - DNA, 20 bp, Artificial Sequence
  Downstream primer for sg46 identification
tgtttagtctttcaagcagg

SEQ ID NO: 164 - DNA, 20 bp, Artificial Sequence
  Upstream primer for sg47 identification
atgataacaagacctggctg

SEQ ID NO: 165 - DNA, 20 bp, Artificial Sequence
  Downstream primer for sg47 identification
tctttcaagcaggtgattac

SEQ ID NO: 166 - DNA, 20 bp, Artificial Sequence
  Upstream primer for sg48 identification
ataacaagacctggctgagc

SEQ ID NO: 167 - DNA, 20 bp, Artificial Sequence
  Downstream primer for sg48 identification
tcaagcaggtgattacagga

SEQ ID NO: 168 - DNA, 22 bp, Artificial Sequence
  PAH Mut-sgRNA
cctccaacctgtggctggcctg

SEQ ID NO: 169 - DNA, 22 bp, Artificial Sequence
  PAH Rec-sgRNA1
tccgcctccaacctgtggctgg

SEQ ID NO: 170 - DNA, 22 bp, Artificial Sequence
  PAH Rec-sgRNA2
ccgcctccaacctgtggctggc

SEQ ID NO: 171 - DNA, 22 bp, Artificial Sequence
  PAH Rec-sgRNA3
cgcctccaacctgtggctggcc

SEQ ID NO: 172 - DNA, 22 bp, Artificial Sequence
  PAH Rec-sgRNA4
gcctccaacctgtggctggcct

SEQ ID NO: 173 - DNA, 22 bp, Artificial Sequence
  PAH Rec-sgRNA5
cctccaacctgtggctggcctg

SEQ ID NO: 174 - DNA, 22 bp, Artificial Sequence
  PAH Rec-sgRNA6
ctccaacctgtggctggcctgc

SEQ ID NO: 175 - DNA, 22 bp, Artificial Sequence
  PAH Rec-sgRNA7
tccaacctgtggctggcctgct

SEQ ID NO: 176 - DNA, 22 bp, Artificial Sequence
  PAH Rec-sgRNA8
ccaacctgtggctggcctgctt

SEQ ID NO: 177 - DNA, 22 bp, Artificial Sequence
  Upstream primer of PAH amplification
gtccctgggcagttatgtgtac

SEQ ID NO: 178 - DNA, 21 bp, Artificial Sequence
  Downstream primer of PAH amplification
caactggtagctggaggacag

(19) United States
(12) Patent Application Publication
(10) Pub. No.: US 2023/0406893 A1
(43) Pub. Date: Dec. 21, 2023

(54) PAM RESTRICTION-FREE ADENINE BASE EDITOR FUSED PROTEIN AND USE THEREOF

(71) Applicant: NATIONAL RESEARCH INSTITUTE FOR FAMILY PLANNING, Beijing (CN)

(72) Inventors: Xu MA, Beijing (CN); Xiaofang CAO, Beijing (CN); Xiaohua JIN, Beijing (CN)

(73) Assignee: NATIONAL RESEARCH INSTITUTE FOR FAMILY PLANNING, Beijing (CN)

(21) Appl. No.: 18/037,689

(22) PCT Filed: Nov. 17, 2021
(86) PCT No.: PCT/CN2021/131279

§ 371 Date: May 18, 2023

(30) Foreign Application Priority Data
Aug. 10, 2021 (CN) 202110912085.0

(51) Int. Cl. C07K 14/47; C12N 9/22; A61P 3/00; C12N 15/63; C12N 15/10

(52) U.S. Cl. CPC C07K 14/4702; C12N 9/22; A61P 3/00; C12N 15/63; C12N 15/102; C12Y 305/04004

(57) ABSTRACT

Disclosed are a PAM restriction-free adenine base editor fused protein and use. A mutant polypeptide is provided, which comprises an N-terminal fragment of SpRY(D10A), a TadA8e fragment, and a C-terminal fragment of SpRY(D10A) polypeptide in sequence from the N terminus to the C terminus. A fused protein including the mutant polypeptide can target the whole genome, thereby broadening the editable range of the genome. It can induce a base transition of A:T to G:C more efficiently, and has great use potential, including but not limited to, simulation or correction of pathogenic sites in genetic disorders. It lowers off-target at the transcriptome level, and is a mutant form with high efficiency and low off-target.

### FIELD

The present disclosure belongs to the field of biomedicines and relates to a PAM restriction-free adenine base editor fused protein and use thereof.

### BACKGROUND OF THE INVENTION

The CRISPR/Cas9 system, which was originally discovered in bacteria and archaea, has been optimized and modified to form a powerful gene editing tool that is widely used in researches on DNA knockout, knockin, modification, etc. The CRISPR/Cas9 system is composed of two parts, i.e., Cas9 nuclease and sgRNA that recognizes a target sequence. The complementary pairing between the sgRNA and the target sequence mediates the directional cleavage of the genome by Cas9 nuclease to cause double-strand break (DSB) of DNA, and homologous recombination (with a template) and non-homologous end joining (without a template) are performed by the correction mechanism in cells, thereby achieving editing of target sites^{[1, 2]}. Subsequently, David Liu, et al. constructed nickase Cas9 (nCas9) with an inactivated RuvC domain, and developed single base editing systems, i.e., a cytosine base editor (CBE) and an adenine base editor (ABE) on this basis. The two base editors can respectively achieve base transitions of C:G to T:A and A:T to G:C without causing double-strand break of DNA, which greatly improves the efficiency and safety of single base editing^{[2, 3]}.

ABE is formed by fusing adenine deaminase with nCas9. According to data comprised in the ClinVar database, 58% of genetic variations associated with human diseases are point mutations, and 47% of pathogenic point mutations can be corrected through an ABE-induced base transition of A:T to G:C^{[4]}. Numerous studies have shown the use value of ABE in the field of the correction of diseases. For example, ABE and corresponding sgRNA are delivered by a virus to the muscles of a mouse with Duchenne muscular dystrophy to correct a nonsense mutation in the pathogenic gene DMD^{[5]}. ABE in the form of mRNA is delivered by lipid nanoparticles to the liver of an adult mouse with tyrosinemia to correct a pathogenic cleavage site mutation to recover the expression of FAH in liver cells^{[6]}. However, the editing of sites by ABE is restricted by an editing window and a PAM sequence. A PAM sequence recognized by ABEmax that is the most widely applied is NGG. To further broaden an editing range of a base editor, ABEs that recognize different PAM sequences have emerged one after another, such as xABE and ABE-NG recognizing a PAM sequence of NG^{[7]}. Among them, ABEmax-SpRY with the loosest PAM restriction was published in March 2020 and can recognize PAM sequences of NRN (R represents A or G) and NYN (Y represents C or T)^{[8]}. ABEmax-SpRY can target all sequences of the genome, but the editing frequency of ABEmax-SpRY is relatively low. Moreover, the problem of off-target at the transcriptome level of ABE is still unsolved, which limits the use of the base editor. Therefore, it is necessary to improve and optimize ABE.

### SUMMARY

In some embodiments, the present disclosure provides a isolated mutant polypeptide, which comprises an N-terminal fragment of SpRY(D10A), a TadA8e fragment, and a C-terminal fragment of SpRY(D10A) polypeptide in sequence from the N terminus to the C terminus.

In some embodiments, an amino acid sequence of the N-terminal fragment of SpRY(D10A) protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 1, or an amino acid sequence of the TadA8e fragment has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 3, or an amino acid sequence of the C-terminal fragment of SpRY(D10A) protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 5.

In some embodiments, the amino acid sequence of the N-terminal fragment of SpRY(D10A) protein is shown as SEQ ID NO: 1, the amino acid sequence of the TadA8e fragment is shown as SEQ ID NO: 3, and the amino acid sequence of the C-terminal fragment of SpRY(D10A) protein is shown as SEQ ID NO: 5.

In some embodiments, a nucleotide sequence encoding the N-terminal fragment of SpRY(D10A) protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a nucleotide sequence shown as SEQ ID NO: 2.

In some embodiments, the nucleotide sequence encoding the N-terminal fragment of SpRY(D10A) protein is shown as SEQ ID NO: 2.

In some embodiments, a nucleotide sequence encoding the TadA8e fragment has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a nucleotide sequence shown as SEQ ID NO: 4.

In some embodiments, the nucleotide sequence encoding the TadA8e fragment is shown as SEQ ID NO: 4.

In some embodiments, a nucleotide sequence encoding the C-terminal fragment of SpRY(D10A) protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a nucleotide sequence shown as SEQ ID NO: 6.

In some embodiments, the nucleotide sequence encoding the C-terminal fragment of SpRY(D10A) protein is shown as SEQ ID NO: 6.

In some embodiments, the mutant polypeptide is used for gene editing.

In some embodiments, an editing window of the gene editing covers about 3-10 positions.

In some embodiments, the editing window of the gene editing covers about 8-10 positions.

In some embodiments, the mutant polypeptide comprises an amino acid sequence that has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a sequence shown as SEQ ID NO: 13.

In some embodiments, the mutant polypeptide comprises the sequence shown as SEQ ID NO: 13.

In some embodiments, the present disclosure provides a isolated fused protein, which comprises the mutant polypeptide.

In some embodiments, the fused protein including the mutant polypeptide can target the whole genome, thereby broadening the editable range of the genome. It can induce a base transition of A:T to G:C more efficiently, and has great use potential, including but not limited to, the simulation or correction of pathogenic sites in genetic disorders. In some embodiments, the fused protein including the mutant polypeptide broadens a base editing window, reduces off-target on the transcriptome level, and is a mutant form with high efficiency and low off-target.

In some embodiments, compared with the existing adenine base editor mutants, ABEmax-SpRY has no PAM restriction, and effectively increases the targetable range of the genome, but has low editing activity.

In some embodiments, the inventors replace an adenine deaminase dimer in ABEmax-SpRY with adenine deaminase TadA8e in ABE8e to construct 8e-SpRY. Compared with ABEmax-SpRY, 8e-SpRY can not only induce a base transition more efficiently but also broaden a base editing window.

In some embodiments, the inventors also construct 4 mutantsbased on 8e-SpRY, which are CE-8e-SpRY, V106W-SpRY, 8e-SpRY-HF, and V106W-SpRY-HF, respectively. Through a comprehensive assessment of editing frequency and off-target, it is found that CE-8e-SpRY is a mutant form with high efficiency and low off-target.

In some embodiments, the fused protein also comprises a linker peptide, which is located between the N-terminal fragment of the SpRY(D10A) protein and the TadA8e fragment, and/or located between the TadA8e fragment and the C-terminal fragment of SpRY(D10A) protein.

In some embodiments, a sequence of the linker peptide has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 7.

In some embodiments, the amino acid sequence of the linker peptide is shown as SEQ ID NO: 7.

In some embodiments, a nucleotide sequence encoding the linker peptide has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a nucleotide sequence shown as SEQ ID NO: 8.

In some embodiments, the nucleotide sequence encoding the linker peptide is shown as SEQ ID NO: 8.

In some embodiments, the fused protein also comprises a nuclear localization signal fragment.

In some embodiments, the nuclear localization signal fragment is located at the N terminus and/or the C terminus of the fused protein.

In some embodiments, an amino acid sequence of the nuclear localization signal fragment has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 9 and/or SEQ ID NO: 11.

In some embodiments, the amino acid sequence of the nuclear localization signal fragment is shown as SEQ ID NO: 9 and/or SEQ ID NO: 11.

In some embodiments, a nucleotide sequence of a nuclear localization signal has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a nucleotide sequence shown as SEQ ID NO: 10 or 12.

In some embodiments, the nucleotide sequence of the nuclear localization signal is shown as SEQ ID NO: 10 or 12.

In some embodiments, the nuclear localization signal fragment comprises about two copies.

In some embodiments, an amino acid sequence of the fused protein comprises an amino acid sequence that has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 13.

In some embodiments, the amino acid sequence of the fused protein comprises the sequence shown as SEQ ID NO: 13.

In some embodiments, the fused protein can effectively edit mutation sites located at the 3rd position to the 10th position in an editing window.

In some embodiments, the fused protein can effectively edit mutation sites located at the 8th position to the 10th position in the editing window.

In some embodiments, the fused protein can effectively edit a mutation site located at the 10th position in the editing window.

In some embodiments, the fused protein is used for gene editing.

In some embodiments, an editing window of the gene editing covers about 3-10 positions.

In some embodiments, the editing window of the gene editing covers about 8-10 positions.

In some embodiments, the present disclosure provides a polynucleotide encoding the mutant polypeptide or the fused protein, or a complementary sequence thereof.

In some embodiments, the polynucleotide is a nucleic acid construct.

In some embodiments, the present disclosure provides a vector, which comprises the polynucleotide.

In some embodiments, the vector is a recombinant expression vector.

In some embodiments, a skeleton of the vector is selected from pCMV and a derived plasmid thereof.

In some embodiments, the derived plasmid of pCMV comprises ABEmax-SpRY.

In some embodiments, the vector comprises a plasmid or virus vector.

In some embodiments, the vector is a plasmid or virus vector used for expression in higher eukaryotes or prokaryotes.

In some embodiments, the eukaryotes are selected from brain neuroma cells and embryonic kidney cells.

In some embodiments, the human embryonic kidney cells comprise HEK293T cells.

In some embodiments, the brain neuroma cells comprise N2a cells.

In some embodiments, the present disclosure provides a method for producing the vector, by adding a polynucleotide encoding an N-terminal fragment of SpRY(D10A) protein, a polynucleotide encoding a TadA8e fragment, and a polynucleotide encoding a C-terminal fragment of SpRY(D10A) protein to a skeleton plasmid to obtain the vector.

In some embodiments, the vector comprises a plasmid or virus vector.

In some embodiments, the vector is a plasmid or virus vector used for expression in higher eukaryotes or prokaryotes.

In some embodiments, a nucleotide sequence encoding the N-terminal fragment of SpRY(D10A) protein is shown as SEQ ID NO: 2.

In some embodiments, a nucleotide sequence encoding the TadA8e fragment is shown as SEQ ID NO: 4.

In some embodiments, a nucleotide sequence encoding the C-terminal fragment of SpRY(D10A) protein is shown as SEQ ID NO: 6.

In some embodiments, the skeleton plasmid comprises pCMV or a derived plasmid thereof: ABEmax-SpRY.

In some embodiments, the eukaryotes are selected from brain neuroma cells and embryonic kidney cells.

In some embodiments, the human embryonic kidney cells comprise HEK293T cells.

In some embodiments, the brain neuroma cells comprise N2a cells.

In some embodiments, the method comprises removing a TadA fragment from the derived plasmid ABEmax-SpRY and replacing amino acids located at the 1048th site to the 1063rd site in SpRY(D10A) with TadA8e to construct a recombinant expression vector.

In some embodiments, the vector is a CE-8e-SpRY plasmid.

In some embodiments, the present disclosure provides an sgRNA.

In some embodiments, a sequence of the sgRNA comprises the sequence shown in SEQ ID NO: 18 to SEQ ID NO: 65.

In some embodiments, the present disclosure provides an expression system. The expression system comprises the expression vector, or the exogenous polynucleotide is integrated into the genome of the expression system.

In some embodiments, the expression system expresses the fused protein or the exogenous sequence integrated into the genome of the expression system expresses the fused protein, or the expression system expresses a polynucleotide containing the polynucleotide, or the exogenous polynucleotide is integrated into the genome of the expression system.

In some embodiments, the expression system also comprises RNA.

In some embodiments, the RNA is guide RNA.

In some embodiments, the RNA is sgRNA.

In some embodiments, a sequence of the sgRNA comprises a sequence that has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a sequence shown as any one of SEQ ID NO: 18 to SEQ ID NO: 65.

In some embodiments, the sequence of the sgRNA comprises the sequence shown as any one of SEQ ID NO: 18 to SEQ ID NO: 65.

In some embodiments, the present disclosure provides a host cell, which comprises the polynucleotide or the vector, or the expression system.

In some embodiments, the present disclosure provides a composition, which comprises an effective amount of at least one of the mutant polypeptides, the fused protein, the polynucleotide, the vector, and the host cell.

In some embodiments, the composition is a kit.

In some embodiments, the composition also comprises RNA.

In some embodiments, the RNA is guide RNA.

In some embodiments, the RNA is sgRNA.

In some embodiments, a sequence of the sgRNA comprises a sequence that has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a sequence shown as any one of SEQ ID NO: 18 to SEQ ID NO: 65.

In some embodiments, the sequence of the sgRNA comprises the sequence shown as any one of SEQ ID NO: 18 to SEQ ID NO: 65.

In some embodiments, the present disclosure provides use of any one of the mutant polypeptides, the fused protein, the polynucleotide, the vector, the expression system, and the host cell in the preparation of a drug for treating a genetic disorder.

In some embodiments, the present disclosure provides use of any one of the mutant polypeptide, the fused protein, the polynucleotide, the vector, the expression system, and the host cell in the preparation of a gene editing reagent.

In some embodiments, an editing window of the gene editing covers about 3-10 positions.

In some embodiments, the editing window of the gene editing covers about 8-10 positions.

In some embodiments, the present disclosure provides a base editing system, which comprises any one of the mutant polypeptides, the fused protein, the polynucleotide, the vector, the expression system, and the host cell.

In some embodiments, the base editing system also comprises RNA.

In some embodiments, the RNA is guide RNA.

In some embodiments, the RNA is sgRNA.

In some embodiments, the present disclosure provides a gene editing method, and gene editing is performed through the base editing system.

In some embodiments, an editing window of the gene editing covers about 3-10 positions.

In some embodiments, the editing window of the gene editing covers about 8-10 positions.

In some embodiments, the present disclosure provides a method for producing the mutant polypeptide or the fused protein by recombination, which comprises the following steps: introducing the vector into host cells to produce transfected or infected host cells, culturing the transfected or infected host cells in vitro, collecting cell cultures, and optionally purifying produced mutant polypeptides or fused proteins.

In some embodiments, the present disclosure provides a preparation method for the mutant polypeptide or the fused protein, which comprises: (1) adding a polynucleotide encoding the N-terminal fragment of SpRY(D10A) protein, a polynucleotide encoding the TadA8e fragment, and a polynucleotide encoding the C-terminal fragment of SpRY(D10A) protein to a skeleton plasmid to obtain a recombinant expression vector; and (2) transfecting host cells with the recombinant expression vector to enable the host cells to express the mutant polypeptide or the fused protein.

In some embodiments, the nucleotide sequence encoding the N-terminal fragment of SpRY(D10A) protein is shown as SEQ ID NO: 2.

In some embodiments, the nucleotide sequence encoding the TadA8e fragment is shown as SEQ ID NO: 4.

In some embodiments, the nucleotide sequence encoding the C-terminal fragment of SpRY(D10A) protein is shown as SEQ ID NO: 6.

In some embodiments, the skeleton plasmid comprises pCMV or a derived plasmid thereof: ABEmax-SpRY.

In some embodiments, the method comprises removing a TadA dimer from the derived plasmid ABEmax-SpRY, and replacing amino acids at the 1048th site to the 1063rd site in SpRY(D10A) with TadA8e to construct the recombinant expression vector.

In some embodiments, the vector is a plasmid or virus vector.

In some embodiments, the vector is a plasmid or virus vector used for expression in higher eukaryotes or prokaryotes.

In some embodiments, the eukaryotes are selected from brain neuroma cells and embryonic kidney cells.

In some embodiments, the human embryonic kidney cells comprise HEK293T cells.

In some embodiments, the brain neuroma cells comprise N2a cells.

In some embodiments, the present disclosure provides a method for producing the vector, which comprises the steps: introducing the vector into an appropriate cell line, culturing the cell line under appropriate conditions to produce target vectors, collecting produced plasmids from cultures of the cell line, and optionally purifying the plasmids.

In some embodiments, the present disclosure provides a treatment method of a genetic disorder, which comprises the following steps: administering a certain amount of at least one of the mutant polypeptide, the fused protein, and the polynucleotide, or any combination thereof that are effective for a genetic disorder to a subject.

In some embodiments, the genetic disorder comprises phenylketonuria.

In some embodiments, the above protein is a isolated polypeptide.

In some embodiments, the above polypeptide is a isolated polypeptide.

In some embodiments, the above nucleic acid is a isolated nucleic acid.

### BRIEF DESCRIPTION OF THE DRAWINGS

FIG. 1 is a schematic diagram of ABEmax-SpRY, and 8e-SpRY and mutants thereof.

FIG. 2 to FIG. 7 are diagrams of editing frequency of ABEmax-SpRY and 8e-SpRY in a case that PAM is NNN.

FIG. 8 is a diagram of statistical results of multi-point editing frequency of ABEmax-SpRY and 8e-SpRY.

FIG. 9 is a diagram of editing windows of ABEmax-SpRY and 8e-SpRY.

FIG. 10 to FIG. 15 are diagrams of editing frequency of 8e-SpRY and mutants thereof in a case that PAM is NNN.

FIG. 16 is a diagram of statistical results of multi-point editing frequency of 8e-SpRY and mutants thereof.

FIG. 17 is a diagram of statistical results of multi-point editing frequency of 8e-SpRY and mutants thereof in a case that PAM is NAN, NGN, NCN or NTN.

FIG. 18 is a diagram of editing windows of 8e-SpRY and mutants thereof.

FIG. 19 is a diagram of DNA targeting editing frequency of ABEmax-SpRY, and 8e-SpRY and mutants thereof.

FIG. 20 is a diagram of the number of RNA off-target of ABEmax-SpRY, and 8e-SpRY and mutants thereof.

FIG. 21 is a schematic diagram of A-to-I RNA off-target of ABEmax-SpRY, and 8e-SpRY and mutants thereof.

FIG. 22 is a sanger sequencing diagram of a genotype of a PKU 728 G>A cell model and a sanger sequencing diagram of correction efficiency of 8 types of correction sgRNA.

FIG. 23 is a histogram of correction efficiency of 3 types of correction sgRNA. and

FIG. 24 is a sanger sequencing diagram of correction efficiency of 3 other ABE mutants.

### DETAILED DESCRIPTION

The technical solutions of the present disclosure will be further illustrated by specific examples, but the specific examples are not intended to limit the scope of protection of the present disclosure. Some non-essential modifications and adjustments made by others based on the concepts of the present disclosure still fall within the scope of protection of the present disclosure.

Phenylketonuria (PKU) is a kind of congenital metabolic disease, which is caused by a phenylalanine (PA) metabolic disorder due to phenylalanine hydroxylase (PAH) deficiency in liver that is caused by a chromosomal gene mutation.

### Example 1 Construction of Base Editor Plasmids

First, 8e-SpRY and corresponding mutants were constructed. Primers were designed according to instructions of ClonExpress MultiS One Step Cloning Kit (Vazyme, C113-01), and used to amplify a TadA8e fragment in ABE8e (Addgene, #138489), and a TadA dimer in ABEmax-SpRY (Addgene, #140003) was replaced with TadA8e to construct an 8e-SpRY plasmid.

TadA8e in 8e-SpRY was deleted from its original site, and amino acids located at the 1048th site to the 1063rd site in SpRY(D10A) were replaced with TadA8e to construct a CE-8e-SpRY plasmid, which comprised the N terminus of SpRY(D10A), TadA8e, and the C terminus of SpRY(D10A) in sequence from the 5′ end to the 3′ end. A nucleotide sequence of the N terminus of SpRY(D10A) is shown as SEQ ID NO: 2 (an amino acid sequence is shown as SEQ ID NO: 1), a nucleotide sequence of TadA8e is shown as SEQ ID NO: 4 (an amino acid sequence is shown as SEQ ID NO: 3), and a nucleotide sequence of the C terminus of SpRY(D10A) is shown as SEQ ID NO: 6 (an amino acid sequence is shown as SEQ ID NO: 5).

A V106W mutation was performed on TadA8e in 8e-SpRY to obtain V106W-SpRY. A nucleotide sequence of TadA8e V106W is shown as SEQ ID NO: 15, and a nucleotide sequence of SpRY(D10A) is shown as SEQ ID NO: 16.

N497A, R661A, Q695A, and Q926A mutations were performed on SpRY(D10A) in 8e-SpRY to obtain 8e-SpRY-HF. A nucleotide sequence of SpRY(D10A)-HF is shown as SEQ ID NO: 17.

A V106W mutation was performed on TadA8e in 8e-SpRY-HF to obtain V106W-SpRY-HF.

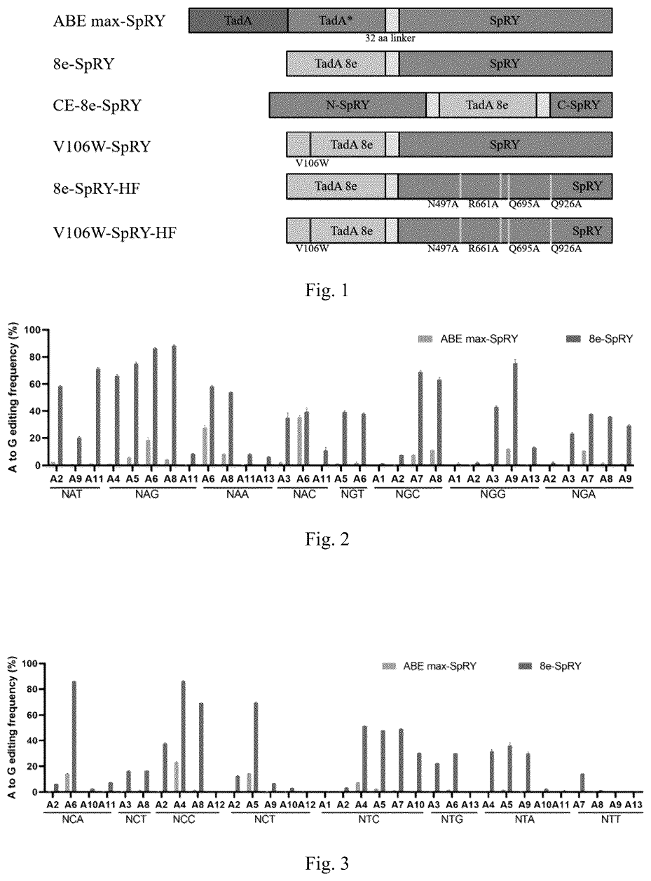

8e-SpRY and the mutants thereof carried nuclear localization signals at both ends, which were bpNLS (a nucleotide sequence of the nuclear localization signal is shown as SEQ ID NO: 10; and an amino acid sequence is shown as SEQ ID NO: 9) or SV40NLS (a nucleotide sequence of the nuclear localization signal is shown as SEQ ID NO: 12; and an amino acid sequence is shown as SEQ ID NO: 11). 8e-SpRY and the mutants thereof are specifically shown in FIG. 1.

(1) ABEmax-SpRY (Fused Protein)

An amino acid sequence of ABEmax-SpRY (fused protein) is shown as SEQ ID NO: 67, and ABEmax-SpRY comprises bpNLS, a TadA dimer, SpRY(D10A), and bpNLS in sequence from the N terminus to the C terminus. In some examples, the nuclear localization signals carried at both ends may also be SV40NLS.

(2) 8e-SpRY (Fused Protein)

An amino acid sequence of 8e-SpRY is shown as SEQ ID NO: 68, and 8e-SpRY comprises bpNLS, TadA8e, SpRY(D10A), and bpNLS in sequence from the N terminus to the C terminus. In some examples, the nuclear localization signals carried at both ends may also be SV40NLS.

(3) CE-8e-SpRY (Fused Protein)

An amino acid sequence of CE-8e-SpRY (fused protein) is shown as SEQ ID NO: 13 (a nucleotide sequence of the CE-8e-SpRY fused protein is shown as SEQ ID NO: 14), and CE-8e-SpRY comprises bpNLS, an N-terminal fragment of SpRY(D10A), a TadA8e fragment, a C-terminal fragment of SpRY(D10A) polypeptide, and bpNLS in sequence from the N terminus to the C terminus. CE-8e-SpRY also comprises a linker peptide located between the N-terminal fragment of SpRY(D10A) and the TadA8e fragment or located between the TadA8e fragment and the C-terminal fragment of SpRY(D10A), and an amino acid sequence of the linker peptide is shown as SEQ ID NO: 7 (a nucleotide sequence encoding the linker peptide in CE-8e-SpRY is shown as SEQ ID NO: 8). In some examples, the nuclear localization signals carried at both ends may also be SV40NLS.

(4) V106W-SpRY (Fused Protein)

An amino acid sequence of V106W-SpRY (fused protein) is shown as SEQ ID NO: 69, V106W-SpRY comprises bpNLS, TadA8eV106W, SpRY(D10A), and bpNLS in sequence from the N terminus to the C terminus, and the nuclear localization signals carried at both ends may also be SV40NLS.

(5) 8e-SpRY-HF (Fused Protein)

An amino acid sequence of 8e-SpRY-HF (fused protein) is shown as SEQ ID NO: 70, 8e-SpRY-HF comprises bpNLS, TadA8e, SpRY(D10A)-HF, and bpNLS in sequence from the N terminus to the C terminus, and the nuclear localization signals carried at both ends may also be SV40NLS.

(6) V106W-SpRY-HF

An amino acid sequence of V106W-SpRY-HF (fused protein) is shown as SEQ ID NO: 71, V106W-SpRY-HF comprises bpNLS, TadA8eV106W, SpRY(D10A)-HF, and bpNLS in sequence from the N terminus to the C terminus, and the nuclear localization signals carried at both ends may also be SV40NLS.

### Example 2

In this example, ABEmax-SpRY, and 8e-SpRY and the mutants thereof were used to edit endogenous sits in 293T cells.

2.1 Construction of sgRNA Plasmids

48 types of sgRNA were designed according to the PAM characteristics of SpRY nuclease with reference to the human genome sequence, which covered 16 different PAM sequences. sgRNA sequences are shown as SEQ ID NO: 18 to SEQ ID NO: 65. The sgRNA sequence added with ACCG at the 5′ end was taken as an upstream sequence, an sgRNA reverse complementary sequence added with AAAC at the 5′ end was taken as a downstream sequence, and after oligo was synthesized, the upstream and downstream sequences were annealed (the program was that: 95° C., 5 min; 95° C.-85° C. at −2° C./s; 85° C.-25° C. at −0.1° C./s; hold at 16° C.) and linked to a pGL3-U6-sgRNA vector (Addgene, #51133) that was cleaved by BsaI (NEB, R3733L). An enzyme cleavage system was: 2 μg of pGL3-U6-sgRNA, 6 μL of CutSmart buffer (NEB, B7204S), 1 μL of BsaI, and ddH2O supplemented to 60 μL and digested overnight at 37° C. A linking system was: 3 μL of Solution I (Takara, 6022Q), 1 μL of enzyme-cleaved vector, and 6 μL of annealing product. and the annealing product and the enzyme-cleaved vector were linked at 16° C. for 30 min, transfected, selected, and identified. Positive clones were shaken to extract plasmids (Axygene, AP-MN-P-250G), and the concentration was measured for later use.

2.2 Culture and Transfection of Cells

HEK293T cells (ATCC) were inoculated and cultured in DMEM (Gibco, C11995500BT) that was added with 10% serum (Gibco, 10270-106) and contained 1% double-antibody (v/v) (Gibco, 15140122). One day before transfection, the cells were placed in a 24-well plate to enable the cell density during transfection to be about 80%, and the medium was replaced 2 h before transfection. The cells in each well were transfected with 600 ng of base editor plasmids and 300 ng of sgRNA plasmids (sequences of sgRNA1 to sgRNA48 are shown as SEQ ID NO: 18 to SEQ ID NO: 65), the plasmids were diluted with 40 μL of DMEM, 3 μL of EZ Trans cell transfection reagent (Life-iLab, AC04L092) was diluted with 40 μL of DMEM, the diluted EZ transfection reagent was added to and uniformly mixed with the diluted plasmids, and the mixture was placed at the room temperature for 15 min. The DMEM mixed with the plasmids and EZ was placed in the 24-well plate and replaced with a complete medium containing 10% serum after 6 h, the expression of green fluorescent protein (GFP) was observed under a microscope after 48 h of transfection, and GFP-positive cells were sorted by using a flow cell sorter.

GFP was carried on the pGL3-U6-sgRNA vector.

2.3 Testing of Editing Frequency

The GFP-positive cells that were obtained by sorting were centrifuged, a supernatant was removed, a lysis solution (including 50 mM KCl, 1.5 mM MgCl₂, 10 mM Tris (pH=8.0), 0.5% Nonidet P-40, 0.5% Tween 20, and 100 μg/mL protease K) was added, a target sequence was amplified by taking the GFP-positive cell lysate as a template, an amplification system comprised 25 μL of 2× buffer (Vazyme, P505), 1 μL of dNTP, 1 μL of Forward Primer (10 μmol/L), 1 μL of Reverse Primer (10 pmol/L), 1 μL of cell lysate, 0.5 μL of DNA polymerase (Vazyme, P505), ddH2O supplemented to 50 μL. Sequences of Forward Primer and Reverse Primer are shown as SEQ ID NO: 72 to SEQ ID NO: 167 (respectively corresponding to sgRNA1 to sgRNA48).

The PCR amplification product was purified by using an extraction kit (Axygen, AP-PCR-250G). The specific procedure was that: PCR-A with a volume 3 times that of the amplification product was added to and uniformly mixed with the amplification product, the mixture was placed in an adsorption column, the adsorption column was centrifuged at 12000 r/min for 1 min, an effluent was discarded, 700 μL of W2 (added with a specified volume of ethanol) was placed in the adsorption column, the adsorption column was centrifuged at 12000 r/min for 1 min, an effluent was discarded, 400 μL of W2 (added with a specified volume of ethanol) was placed in the adsorption column, the adsorption column was centrifuged at 12000 r/min for 1 min, an effluent was discarded, the adsorption column was centrifuged at 12000 r/min for 2 min and uncovered to air-dry the ethanol, 28 μL of ddH2O was placed in the adsorption column, the adsorption column was centrifuged at 12000 r/min for 1 min and eluted, and the purified PCR product was sent to Sanger for sequencing or in-depth testing to analyze an editing effect.

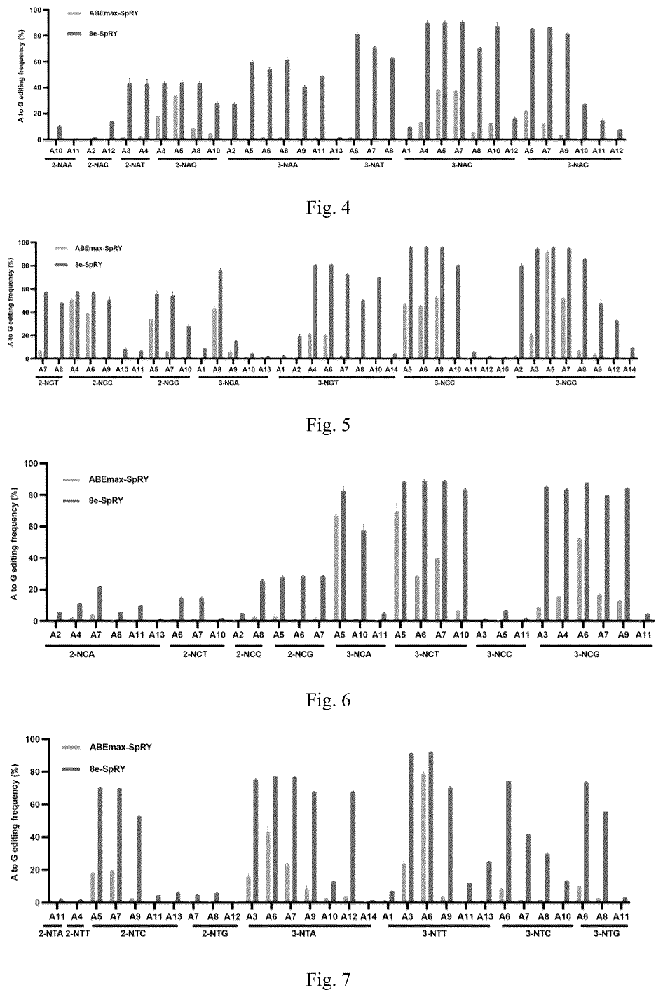

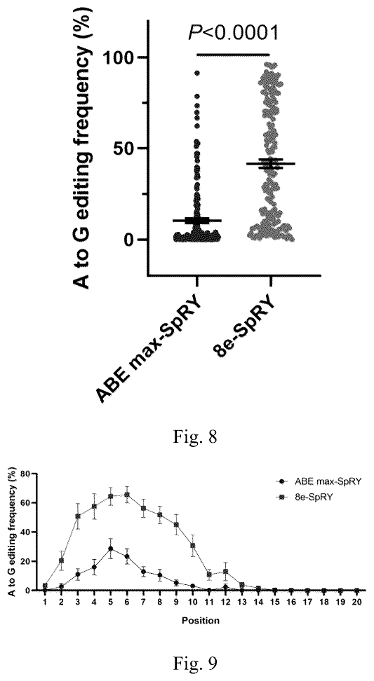

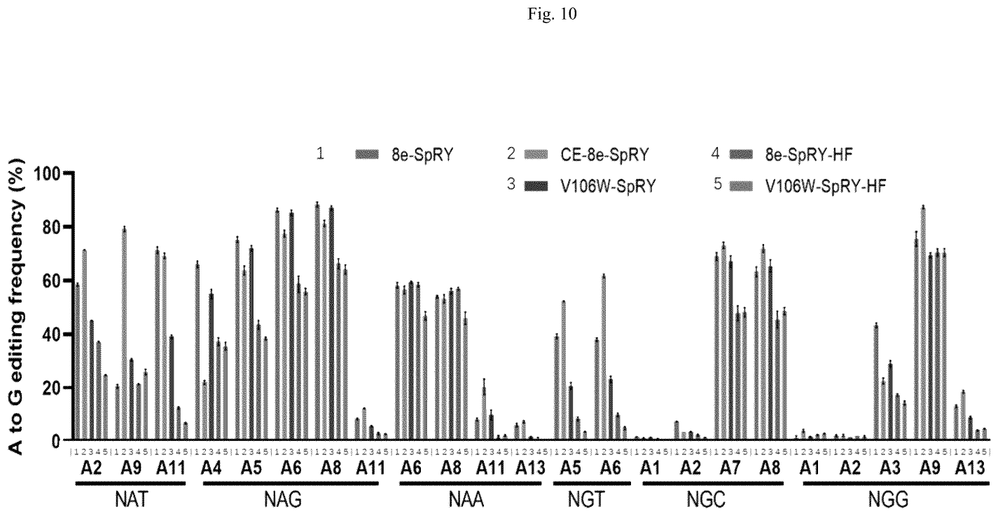

Relevant results are shown in FIG. 2 to FIG. 9. The results show that at all testing sites, covering PAM sequences of NAN, NGN, NCN, and NTN, the editing frequency of 8e-SpRY is obviously higher than that of ABEmax-SpRY. Statistical results of the multi-point editing frequency in FIG. 8 show that 8e-SpRY significantly increases the A-to-G editing frequency. Results of editing windows in FIG. 9 show that a base editing window of ABEmax-SpRY covers 5 or 6 positions, and a base editing window of 8e-SpRY covers 3-10 positions, which is wider.

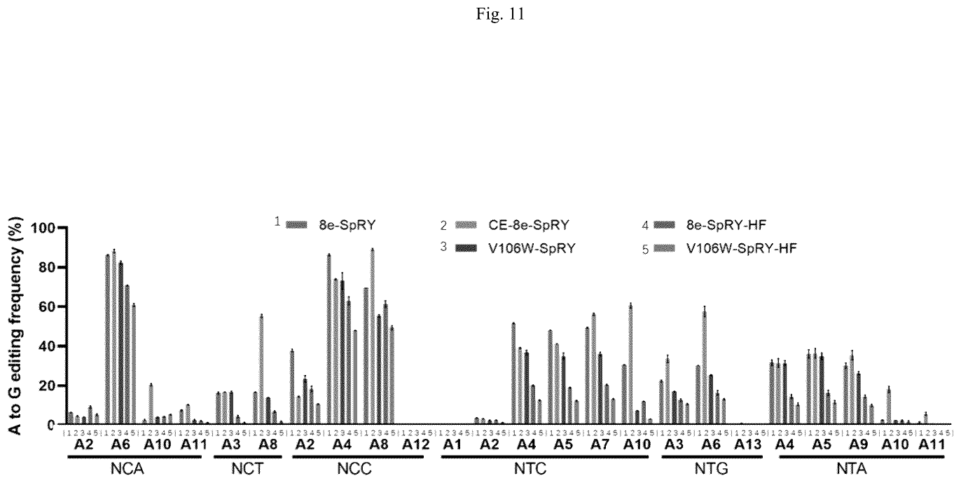

FIG. 10 to FIG. 15 show comparison results of the editing frequency of the mutants of 8e-SpRY in a case that a PAM sequence is NRN (R represents A or G) or NYN (Y represents C or T). CE-8e-SpRY obtained by inserting 8e into the middle of SpRY can well retain the A-to-G editing activity of SpRY, V106W-SpRY obtained by introducing V106W into Tad8e also does not obviously reduce the original editing activity, while 8e-SpRY-HF or V106W-SpRY-HF obtained by introducing 4 mutations into SpRY significantly reduces the editing activity.

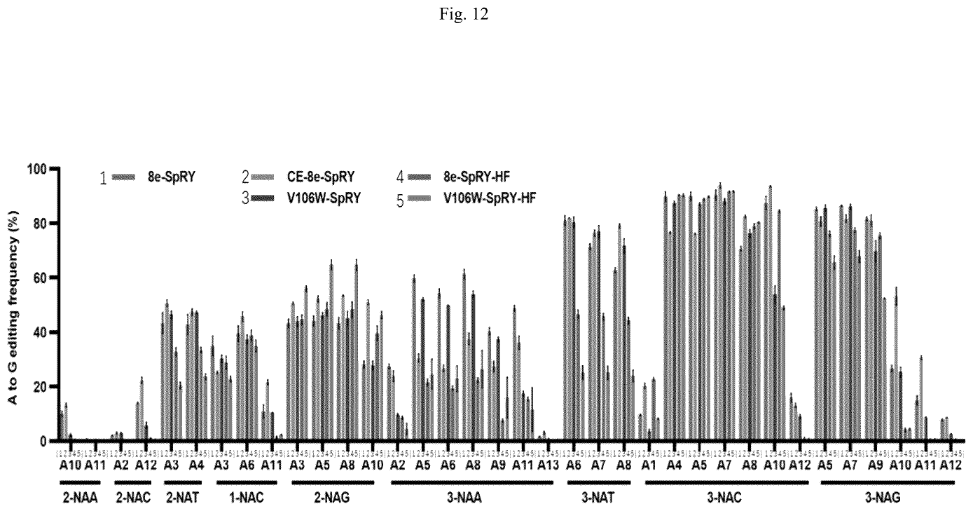

Statistical results of the multi-point editing frequency in FIG. 16 show that 8e-SpRY-HF and V106W-SpRY-HF significantly reduce the activity, the editing frequency of CE-8e-SpRY is increased without a significant difference, and the editing frequency of V106W-SpRY is reduced without a significant difference.

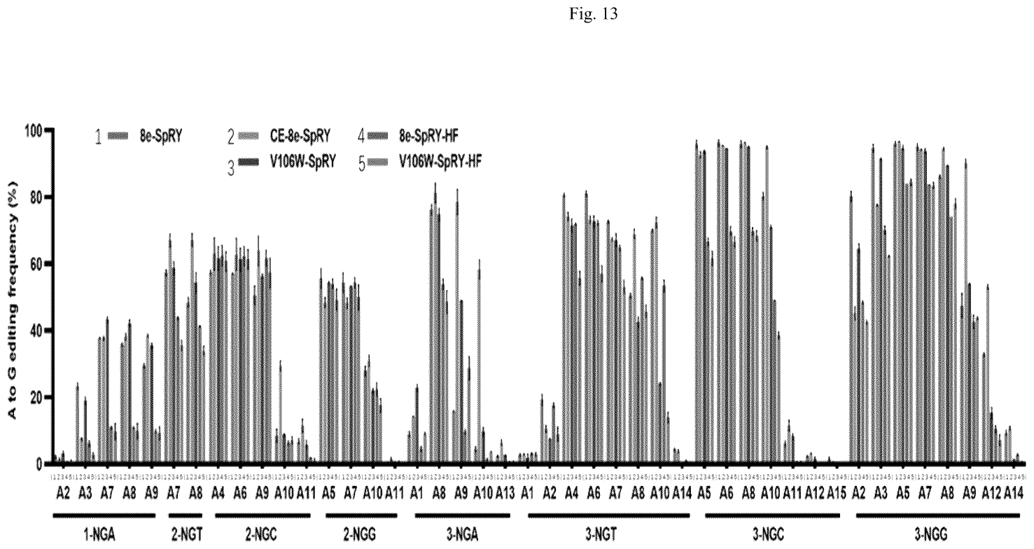

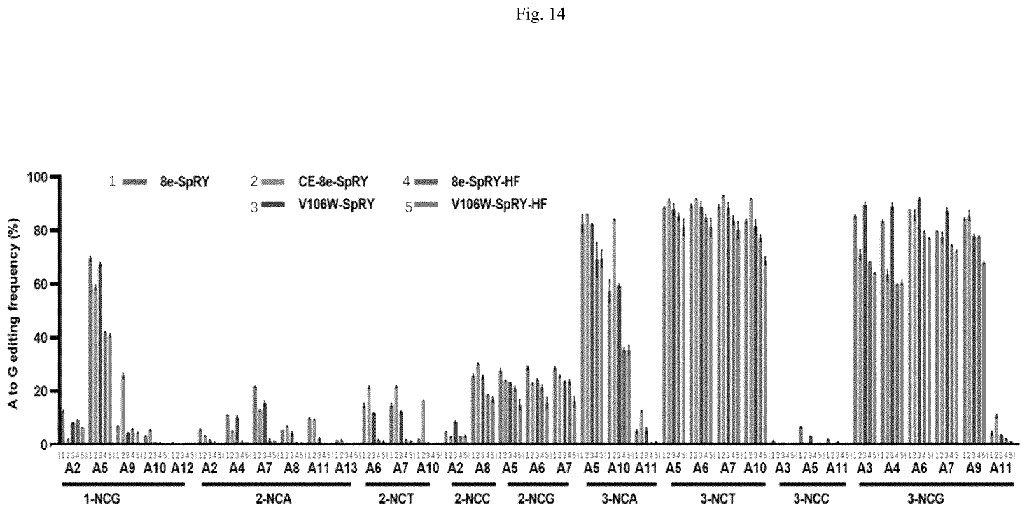

Statistical results of the multi-point editing frequency for NAN, NGN, NCN, and NTN in FIG. 17 show that the editing frequency of CE-8e-SpRY for NGN or NTN is increased, and the editing frequency of V106W-SpRY for 4 PAM sequences is reduced without a statistical significance. Results of editing windows in FIG. 18 show that V106W-SpRY retains the same editing window as 8e-SpRY, which covers 3-10 positions, a highly active editing window (with an editing frequency greater than 40%) covers 3-9 positions, CE-8e-SpRY retains the same editing window, which covers 3-10 positions, a highly active editing window (with the editing frequency greater than 40%) covers 3-10 positions, and the editing frequency of CE-8e-SpRY having an editing window of 8-10 positions is higher than that 8e-SpRY.

TABLE 1
Plasmid combinations used for transfection of cells in Example 2 (1)

| Base editor protein | | sgRNA No. | Result |
|---|---|---|---|
| ABEmax-SpRY or | 1 | SEQ ID NO: 18 | FIG. 4 2-NAA |
| 8e-SpRY | 2 | SEQ ID NO: 19 | FIG. 2 NAT |
| | 3 | SEQ ID NO: 20 | FIG. 4 2-NAC |
| | 4 | SEQ ID NO: 21 | FIG. 2 NAG |
| | 5 | SEQ ID NO: 22 | FIG. 7 2-NTA |
| | 6 | SEQ ID NO: 23 | FIG. 7 2-NTT |
| | 7 | SEQ ID NO: 24 | FIG. 3 NTC |
| | 8 | SEQ ID NO: 25 | FIG. 3 NTG |
| | 9 | SEQ ID NO: 26 | FIG. 3 NCA |
| | 10 | SEQ ID NO: 27 | FIG. 3 NCT |
| | 11 | SEQ ID NO: 28 | FIG. 3 NCC |
| | 12 | SEQ ID NO: 29 | FIG. 3 NCG |
| | 13 | SEQ ID NO: 30 | / |
| | 14 | SEQ ID NO: 31 | FIG. 2 NGT |
| | 15 | SEQ ID NO: 32 | FIG. 2 NGC |
| | 16 | SEQ ID NO: 33 | FIG. 2 NGG |
| | 17 | SEQ ID NO: 34 | FIG. 2 NAA |
| | 18 | SEQ ID NO: 35 | FIG. 4 2-NAT |
| | 19 | SEQ ID NO: 36 | FIG. 2 NAC |
| | 20 | SEQ ID NO: 37 | FIG. 4 2-NAG |
| | 21 | SEQ ID NO: 38 | FIG. 3 NTA |
| | 22 | SEQ ID NO: 39 | FIG. 3 NTT |
| | 23 | SEQ ID NO: 40 | FIG. 7 2-NTC |
| | 24 | SEQ ID NO: 41 | FIG. 7 2-NTG |
| | 25 | SEQ ID NO: 42 | FIG. 6 2-NCA |
| | 26 | SEQ ID NO: 43 | FIG. 6 2-NCT |
| | 27 | SEQ ID NO: 44 | FIG. 6 2-NCC |
| | 28 | SEQ ID NO: 45 | FIG. 6 2-NCG |
| | 29 | SEQ ID NO: 46 | FIG. 2 NGA |
| | 30 | SEQ ID NO: 47 | FIG. 5 2-NGT |
| | 31 | SEQ ID NO: 48 | FIG. 5 2-NGC |
| | 32 | SEQ ID NO: 49 | FIG. 5 2-NGG |
| | 33 | SEQ ID NO: 50 | FIG. 4 3-NAA |
| | 34 | SEQ ID NO: 51 | FIG. 4 3-NAT |
| | 35 | SEQ ID NO: 52 | FIG. 4 3-NAC |
| | 36 | SEQ ID NO: 53 | FIG. 4 3-NAG |
| | 37 | SEQ ID NO: 54 | FIG. 7 3-NTA |
| | 38 | SEQ ID NO: 55 | FIG. 7 3-NTT |
| | 39 | SEQ ID NO: 56 | FIG. 7 3-NTC |
| | 40 | SEQ ID NO: 57 | FIG. 7 3-NTG |
| | 41 | SEQ ID NO: 58 | FIG. 6 3-NCA |
| | 42 | SEQ ID NO: 59 | FIG. 6 3-NCT |
| | 43 | SEQ ID NO: 60 | FIG. 6 3-NCC |
| | 44 | SEQ ID NO: 61 | FIG. 6 3-NCG |
| | 45 | SEQ ID NO: 62 | FIG. 5 3-NGA |
| | 46 | SEQ ID NO: 63 | FIG. 5 3-NGT |
| | 47 | SEQ ID NO: 64 | FIG. 5 3-NGC |
| | 48 | SEQ ID NO: 65 | FIG. 5 3-NGG |

TABLE 2
Plasmid combinations used for transfection of cells in Example 2 (2)

| Base editor protein | | sgRNA | Result |
|---|---|---|---|
| 8e-SpRY or | 1 | SEQ ID NO: 18 | FIG. 12 2-NAA |
| CE-8e-SpRY | 2 | SEQ ID NO: 19 | FIG. 10 NAT |
| or | 3 | SEQ ID NO: 20 | FIG. 12 2-NAC |
| V106W-SpRY | 4 | SEQ ID NO: 21 | FIG. 10 NAG |
| or | 5 | SEQ ID NO: 22 | FIG. 15 2-NTA |
| 8e-SpRY-HF | 6 | SEQ ID NO: 23 | FIG. 15 2-NTT |
| or | 7 | SEQ ID NO: 24 | FIG. 11 NTC |
| V106W-SpRY-HF | 8 | SEQ ID NO: 25 | FIG. 11 NTG |
| | 9 | SEQ ID NO: 26 | FIG. 11 NCA |
| | 10 | SEQ ID NO: 27 | FIG. 11 NCT |
| | 11 | SEQ ID NO: 28 | FIG. 11 NCC |
| | 12 | SEQ ID NO: 29 | FIG. 14 1-NCG |
| | 13 | SEQ ID NO: 30 | / |
| | 14 | SEQ ID NO: 31 | FIG. 10 NGT |
| | 15 | SEQ ID NO: 32 | FIG. 10 NGC |
| | 16 | SEQ ID NO: 33 | FIG. 10 NGG |
| | 17 | SEQ ID NO: 34 | FIG. 10 NAA |
| | 18 | SEQ ID NO: 35 | FIG. 12 2-NAT |
| | 19 | SEQ ID NO: 36 | FIG. 12 1-NAC |
| | 20 | SEQ ID NO: 37 | FIG. 12 2-NAG |
| | 21 | SEQ ID NO: 38 | FIG. 11 NTA |
| | 22 | SEQ ID NO: 39 | FIG. 15 1-NTT |
| | 23 | SEQ ID NO: 40 | FIG. 15 2-NTC |
| | 24 | SEQ ID NO: 41 | FIG. 15 2-NTG |
| | 25 | SEQ ID NO: 42 | FIG. 14 2-NCA |
| | 26 | SEQ ID NO: 43 | FIG. 14 2-NCT |
| | 27 | SEQ ID NO: 44 | FIG. 14 2-NCC |
| | 28 | SEQ ID NO: 45 | FIG. 14 2-NCG |
| | 29 | SEQ ID NO: 46 | FIG. 13 1-NGA |
| | 30 | SEQ ID NO: 47 | FIG. 13 2-NGT |
| | 31 | SEQ ID NO: 48 | FIG. 13 2-NGC |
| | 32 | SEQ ID NO: 49 | FIG. 13 2-NGG |
| | 33 | SEQ ID NO: 50 | FIG. 12 3-NAA |
| | 34 | SEQ ID NO: 51 | FIG. 12 3-NAT |
| | 35 | SEQ ID NO: 52 | FIG. 12 3-NAC |
| | 36 | SEQ ID NO: 53 | FIG. 12 3-NAG |
| | 37 | SEQ ID NO: 54 | FIG. 15 3-NTA |
| | 38 | SEQ ID NO: 55 | FIG. 15 3-NTT |
| | 39 | SEQ ID NO: 56 | FIG. 15 3-NTC |
| | 40 | SEQ ID NO: 57 | FIG. 15 3-NTG |
| | 41 | SEQ ID NO: 58 | FIG. 14 3-NCA |
| | 42 | SEQ ID NO: 59 | FIG. 14 3-NCT |
| | 43 | SEQ ID NO: 60 | FIG. 14 3-NCC |
| | 44 | SEQ ID NO: 61 | FIG. 14 3-NCG |
| | 45 | SEQ ID NO: 62 | FIG. 13 3-NGA |
| | 46 | SEQ ID NO: 63 | FIG. 13 3-NGT |
| | 47 | SEQ ID NO: 64 | FIG. 13 3-NGC |
| | 48 | SEQ ID NO: 65 | FIG. 13 3-NGG |

### Example 3

In this example, results of RNA off-target of ABEmax-SpRY, and 8e-SpRY and the mutants thereof in 293T cells were compared.

3.1 Construction of sgRNA

A sgRNA sequence used for testing RNA off-target was 5′-CTGGAACACAAAGCATAGAC-′3 (SEQ ID NO: 66), which was constructed by the plasmid construction method described in 2.1.

3.2 Culture and Transfection of Cells

The cells were cultured by the method described in 2.2. One day before transfection, the 293T cells were placed in a 6 cm dish to enable the cell density during transfection to be about 80%. The cells in each dish were transfected with 4 μg of base editor plasmids and 2 μg of sgRNA plasmids. The plasmids were diluted with 250 μL of DMEM, 18 μL of EZ Trans cell transfection reagent (Life-iLab, AC04L092) was diluted with 250 μL of DMEM, the diluted EZ transfection reagent was added to and uniformly mixed with the diluted plasmids, and the mixture was placed at the room temperature for 15 min. The DMEM mixed with the plasmids and EZ was placed in the 6 cm dish and replaced with a complete medium containing 10% serum (DMEM+10% FBS) after 6 h, the expression of GFP (on the pGL3-U6-sgRNA vector) was observed under the microscope after 48 h of transfection, and GFP-positive cells were sorted by using the flow cell sorter. A small number of positive cells was used for testing the editing frequency by the method described in 2.3, and the rest of positive cells were used for extraction of RNA that was sent to RNA-Seq.

3.3 Extraction of RNA

The GFP-positive cells were centrifuged at 3000 r/min for 10 min, supernatant was removed, and 1 mL of RNA isolater Total RNA extraction Reagent (Vazyme, R401-01-AA) was added to fully lyse the cells. 200 μL of chloroform was added, and the mixture was shaken violently up and down until uniform, placed at room temperature for 3 min, and centrifuged at 12000 r/min and 4° C. for 15 min. 500 μL of the upper aqueous phase was collected and added with 500 μL of isopropanol, and the mixture was mixed upside down until uniform and centrifuged at 12000 r/min and 4° C. for 15 min. A supernatant was removed, 1 mL of 75% ethanol was added, the mixture was gently inverted several times to wash precipitates, and centrifuged at 12000 r/min and 4° C. for 5 min. A supernatant was removed, and the tube was uncovered to dry the mixture for 5-10 min after the ethanol was completely evaporated, 15 μL of RNase-Free water was added to dissolve the precipitates, and 1 μL of the solution was used for measurement of the concentration. 1 μg of RNA was sent to RNA-Seq.

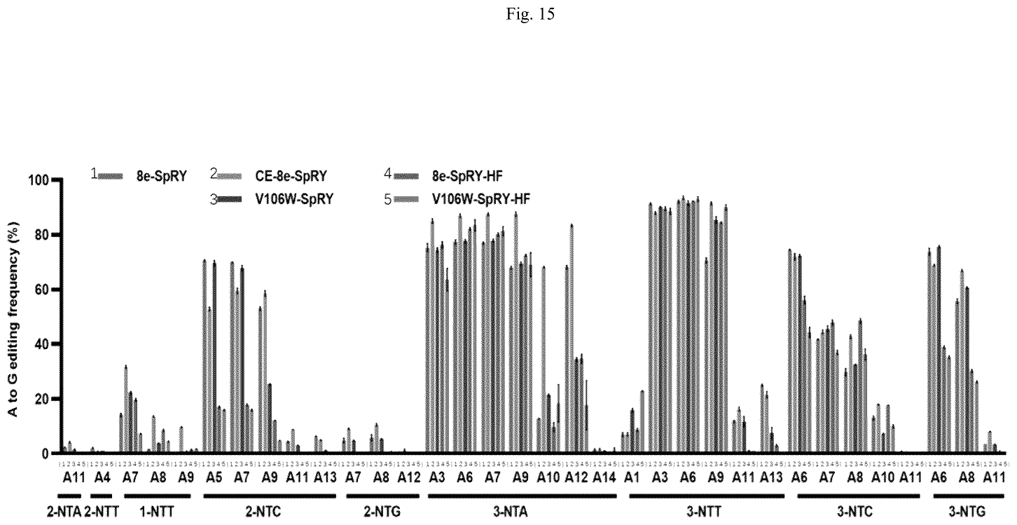

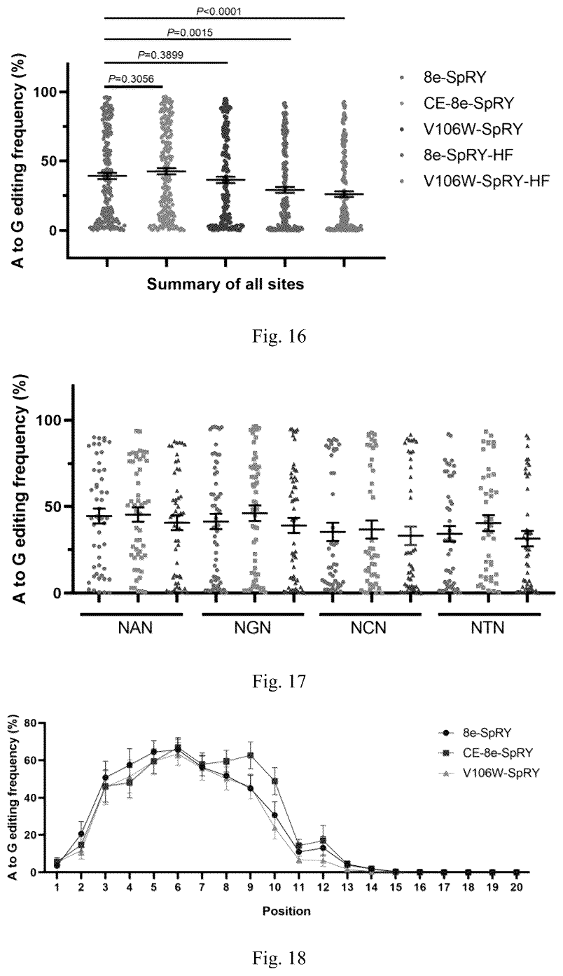

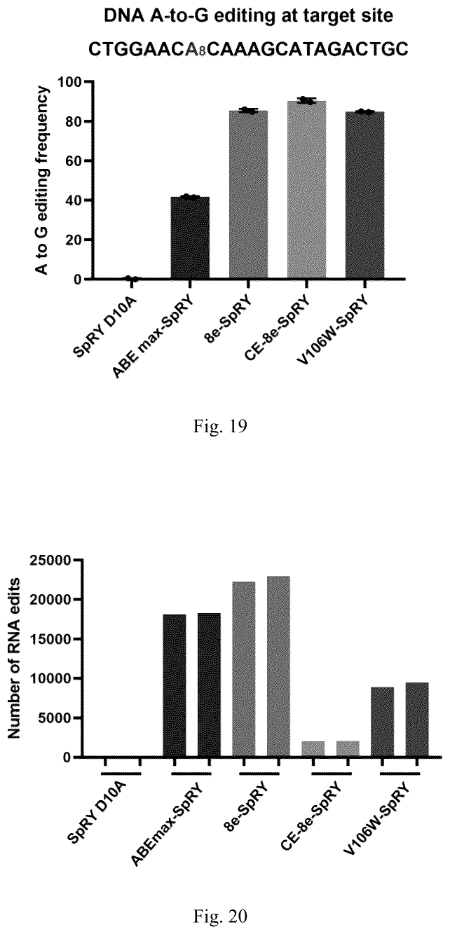

Relevant results are shown in FIG. 19 to FIG. 21. FIG. 19 shows the editing frequency for the 8th A at the target site in DNA, ABEmax-SpRY, and 8e-SpRY and the mutants thereof can induce effective editing, the DNA targeting editing frequency of 8e-SpRY is equivalent to that of the mutants of 8e-SpRY, while the editing frequency of ABEmax-SpRY is relatively low. Results of RNA off-target in FIG. 20 and FIG. 21 show that compared with ABEmax-SpRY and other mutants of 8e-SpRY, CE-8e-SpRY effectively reduces off-target editing at the transcriptome level.

Through comprehensive analysis of the editing frequency test and off-target test results, the inventors have found that the CE-8e-SpRY base editor can target the whole genome, significantly increases the A-to-G editing frequency, effectively reduces the off-target editing at the transcriptome level, and has great use potential.

TABLE 3
Plasmid combinations used for transfection of cells in Example 3

| Base editor protein | sgRNA | Result |
|---|---|---|
| SpRY D10A | SEQ ID NO: 66 | FIG. 19 to FIG. 21 |
| ABEmax-SpRY | SEQ ID NO: 66 | |
| 8e-SpRY | SEQ ID NO: 66 | |
| CE-8e-SpRY | SEQ ID NO: 66 | |
| V106W-SpRY | SEQ ID NO: 66 | |

### Example 4 Use of CE-8e-SpRY in Correction of Pathogenic Sites in a Disease

4.1 Construction of Human PAH 728 G>A Cell Models

4.1.1 Construction of Mutant Mut-sgRNA

mut-sgRNA (shown as SEQ ID NO: 168) was designed and constructed by the plasmid construction method described in 2.1 according to the human genome sequence.

4.1.2 Culture and Transfection of Cells

Cells were cultured by the method described in 2.2. One day before transfection, the cells were placed in a 24-well plate to enable the cell density during transfection to be about 80%, and the medium was replaced 2 h before transfection. The cells in each well were transfected with 600 ng of base editor plasmids and 300 ng of sgRNA plasmids. The plasmids were diluted with 40 μL of DMEM, 3 μL of EZ Trans cell transfection reagent (Life-iLab, AC04L092) was diluted with 40 μL of DMEM, the diluted EZ transfection reagent was added to and uniformly mixed with the diluted plasmids, and the mixture was placed at room temperature for 15 min. The DMEM mixed with the plasmids and EZ was placed in the 24-well plate and replaced with a complete medium containing 10% serum after 6 h, GFP-positive cells were sorted by using the flow cell sorter after 48 h of transfection and placed in a 96-well plate according to 1 positive cell per well, and the 96-well plate was placed in an incubator, the cells were cultured for 14 d, and a genotype of the monoclonal cell was identified.

4.1.3 Identification of a Genotype of the Monoclonal Cell

A part of the monoclonal cells in each well was collected, centrifuged, and added with a lysis solution (including 50 mM KCl, 1.5 mM MgCl₂, 10 mM Tris (pH=8.0), Nonidet P-40, 0.5% Tween 20, and 100 μg/mL protease K), a target sequence was amplified by taking the cell lysate as a template, an amplification system was 25 μL of 2× buffer (Vazyme, P505), 1 μL of dNTP, 1 μL of Forward Primer (10 μmol/L), 1 μL of Reverse Primer (10 pmol/L), 1 μL of cell lysis product, 0.5 μL of DNA polymerase (Vazyme, P505), and ddH2O supplemented to 50 μL. A sequence of Forward Primer is 5′-gtccctgggcagttatgtgtac-3′ (SEQ ID NO: 177), and a sequence of Reverse Primer is 5′-caactggtagctggaggacag-3′ (SEQ ID NO: 178). The amplification product was sent to Sanger for sequencing, and PAH 728 G>A pure and mutant cells were selected, i.e., human PAH 728 G>A cell models.

4.2 Correction of PAH 728 G>A Mutation

CE-8e-SpRY has relatively high editing frequency in a case that an editing window covers 3-10 positions, and can recognize a PAM sequence of NNN. According to the editing window and PAM characteristics of CE-8e-SpRY, the inventors designed 8 types of Rec-sgRNA (shown as SEQ ID NO: 169 to SEQ ID NO: 176) for the pathogenic mutation to be corrected, and constructed the plasmids by the plasmid construction method described in 2.1. Cells were transfected by the cell culture and transfection method described in 2.2. The correction efficiency was tested by the editing frequency test method described in 2.3.

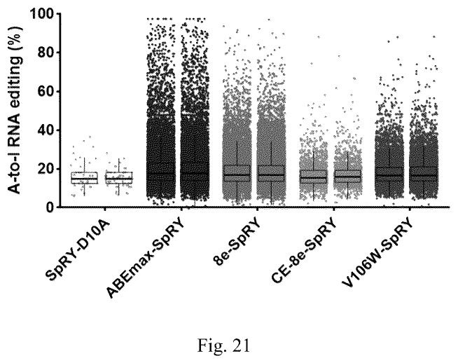

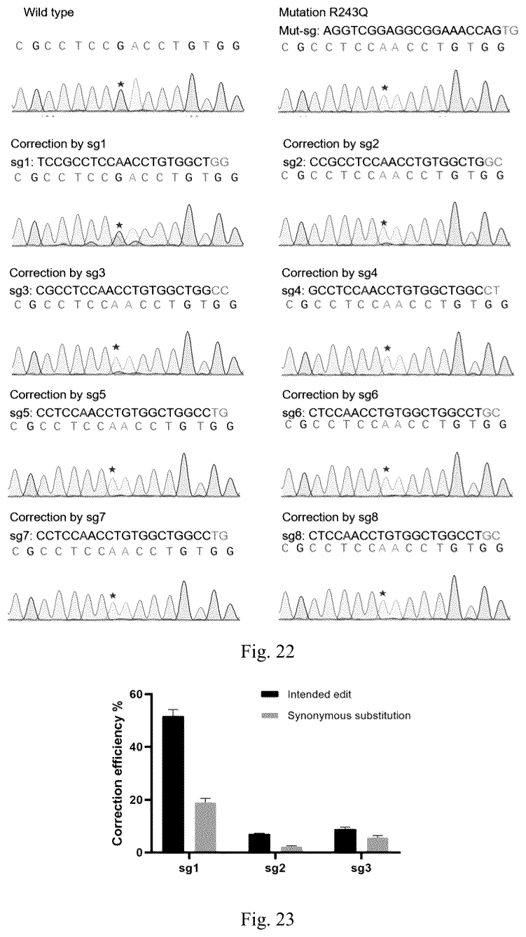

Results are shown in FIG. 22 and FIG. 23. Mut-sgRNA successfully induces a 728 G>A pure mutation. Among the 8 types of Rec-sgRNA, Rec-sgRNA1 (i.e., sg1 in FIG. 22 and FIG. 23) has the highest 728 G>A correction efficiency, and Rec-sgRNA2 (i.e., sg2 in FIG. 22 and FIG. 23) and Rec-sgRNA3 (i.e., sg3 in FIG. 22 and FIG. 23) have weak correction effects.

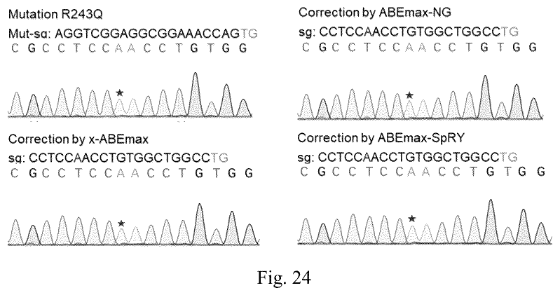

According to the PAM characteristics and editing windows of x-ABEmax, ABEmax-NG, and ABEmax-SpRY, correction sgRNA of the 3 base editors is shown as SEQ ID NO: 173. The sgRNA was constructed by the plasmid construction method described in 2.1, and used to transfect cells by the cell culture and transfection method described in 2.2, and the correction efficiency was tested by the editing frequency test method described in 2.3. Results are shown in FIG. 24. The 3 base editors do not have a significant correction effect on the 728 G>A mutation site.

This example indicates that CE-8e-SpRY recognizes the PAM sequence of NNN, multiple types of sgRNA can be selected for the site to be corrected, and sgRNA that best meets the correction requirements can be selected by screening of sgRNA, which effectively improves a correctable site range and the flexibility of a correction effect. In addition, the 3 existing base editors cannot correct the 728 G>A mutation site within respective editing windows, while CE-8e-SpRY provided by the inventors can effectively edit the mutation site in the case that the mutation site is located at the 10th position in the editing window, which broadens the editable range of the existing base editing tools and shows unique editing characteristics.

### REFERENCES

1. Jinek M, Chylinski K, Fonfara I, et al. A programmable dual-RNA-guided DNA endonuclease in adaptive bacterial immunity. Science. 2012; 337(6096): 816-21.
2. Komor A C, Kim Y B, Packer M S, et al. Programmable editing of a target base in genomic DNA without double-stranded DNA cleavage. Nature. 2016; 533(7603): 420-4.
3. Gaudelli N M, Komor A C, Rees H A, et al. Programmable base editing of A*T to G*C in genomic DNA without DNA cleavage. Nature. 2017; 551(7681): 464-471.
4. Rees H A and Liu D R. Publisher Correction: Base editing: precision chemistry on the genome and transcriptome of living cells. Nat Rev Genet. 2018; 19(12): 801.
5. Ryu S M, Koo T, Kim K, et al. Adenine base editing in mouse embryos and an adult mouse model of Duchenne muscular dystrophy. Nat Biotechnol. 2018; 36(6): 536-539.
6. Song C Q, Jiang T, Richter M, et al. Adenine base editing in an adult mouse model of tyrosinaemia. Nat Biomed Eng. 2020; 4(1): 125-130.
7. Huang T P, Zhao K T, Miller S M, et al. Circularly permuted and PAM-modified Cas9 variants broaden the targeting scope of base editors. Nat Biotechnol. 2019; 37(6): 626-631.
8. Walton R T, Christie K A, Whittaker M N, et al. Unconstrained genome targeting with near-PAMless engineered CRISPR-Cas9 variants. Science. 2020; 368(6488): 290-296.

1-16. (canceled)

17. A isolated mutant polypeptide, characterized by comprising an N-terminal fragment of SpRY(D10A), a TadA8e fragment, and a C-terminal fragment of SpRY(D10A) polypeptide in sequence from the N terminus to the C terminus.

18. The mutant polypeptide according to claim 17, characterized in that an amino acid sequence of the N-terminal fragment of SpRY(D10A) protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 1, or an amino acid sequence of the TadA8e fragment has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 3, or an amino acid sequence of the C-terminal fragment of SpRY(D10A) protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 5,
preferably, a nucleotide sequence encoding the N-terminal fragment of SpRY(D10A) protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a nucleotide sequence shown as SEQ ID NO: 2,
preferably, a nucleotide sequence encoding the TadA8e fragment has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a nucleotide sequence shown as SEQ ID NO: 4,
preferably, a nucleotide sequence encoding the C-terminal fragment of SpRY(D10A) protein has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a nucleotide sequence shown as SEQ ID NO: 6,
preferably, the mutant polypeptide is used for gene editing,
preferably, an editing window of the gene editing covers about 3-10 positions,
preferably, the editing window of the gene editing covers about 8-10 positions, and
preferably, the mutant polypeptide comprises an amino acid sequence that has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a sequence shown as SEQ ID NO: 13.

19. A isolated fused protein, characterized by comprising the mutant polypeptide according to claim 17,
preferably, the fused protein also comprises a linker peptide located between the N-terminal fragment of the SpRY(D10A) protein and the TadA8e fragment, and/or located between the TadA8e fragment and the C-terminal fragment of SpRY(D10A) protein,
preferably, a sequence of the linker peptide has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 7,
preferably, a nucleotide sequence encoding the linker peptide has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a nucleotide sequence shown as SEQ ID NO: 8,
preferably, the fused protein also comprises a nuclear localization signal fragment,
preferably, the nuclear localization signal fragment is located at the N terminus and/or the C terminus of the fused protein,
preferably, an amino acid sequence of the nuclear localization signal fragment has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 9 and/or SEQ ID NO: 11,
preferably, a nucleotide sequence of a nuclear localization signal has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a nucleotide sequence shown as SEQ ID NO: 10 or 12,
preferably, the nuclear localization signal fragment comprising about two copies, preferably, the fused protein comprising an amino acid sequence that has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with an amino acid sequence shown as SEQ ID NO: 13,
preferably, the fused protein is used for gene editing,
preferably, an editing window of the gene editing covers about 3-10 positions,
preferably, the editing window of the gene editing covers about 8-10 positions,
preferably, the fused protein is capable of targeting the whole genome and inducing a base transition of A:T to G:C more efficiently,
preferably, the fused protein is capable of effectively editing mutation sites located at the 3rd position to the 10th position in an editing window,
preferably, the fused protein is capable of effectively editing mutation sites located at the 8th position to the 10th position in the editing window, and
preferably, the fused protein is capable of effectively editing a mutation site located at the position in the editing window.

20. A polynucleotide encoding the mutant polypeptide according to claim 17, or a complementary sequence thereof,
preferably, the polynucleotide is a nucleic acid construct.

21. A polynucleotide encoding the fused protein according to claim 19, or a complementary sequence thereof,
preferably, the polynucleotide is a nucleic acid construct.

22. A vector, characterized by comprising the polynucleotide according to claim 20,
preferably, the vector is a recombinant expression vector,
preferably, a skeleton of the vector is selected from pCMV and a derived plasmid thereof,
preferably, the derived plasmid of pCMV comprises ABEmax-SpRY,
preferably, the vector comprises a plasmid or virus vector,
preferably, the vector is a plasmid or virus vector used for expression in higher eukaryotes or prokaryotes,
preferably, the eukaryotes are selected from brain neuroma cells and embryonic kidney cells,
preferably, the human embryonic kidney cells comprise HEK293T cells, and
preferably, the brain neuroma cells comprise N2a cells.

23. A method for producing the vector according to claim 22, characterized by adding a polynucleotide encoding an N-terminal fragment of SpRY(D10A) protein, a polynucleotide encoding a TadA8e fragment, and a polynucleotide encoding a C-terminal fragment of SpRY(D10A) protein to a skeleton plasmid to obtain the vector,
preferably, the vector comprises a plasmid or virus vector,
preferably, the vector is a plasmid or virus vector used for expression in higher eukaryotes or prokaryotes,
preferably, a nucleotide sequence encoding the N-terminal fragment of SpRY(D10A) protein is shown as SEQ ID NO: 2,
preferably, a nucleotide sequence encoding the TadA8e fragment is shown as SEQ ID NO: 4,
preferably, a nucleotide sequence encoding the C-terminal fragment of SpRY(D10A) protein is shown as SEQ ID NO: 6,
preferably, the skeleton plasmid comprises pCMV or a derived plasmid thereof: ABEmax-SpRY,
preferably, the eukaryotes is selected from brain neuroma cells and embryonic kidney cells,
preferably, the human embryonic kidney cells comprise HEK293T cells,
preferably, the brain neuroma cells comprise N2a cells,
preferably, the method comprising removing a TadA fragment from the derived plasmid ABEmax-SpRY, and replacing amino acids located at the 1048th site to the 1063rd site in SpRY(D10A) with TadA8e to construct a recombinant expression vector, and
preferably, the vector is a CE-8e-SpRY plasmid.

24. An expression system, characterized in that the expression system expresses the fused protein according to claim 19, or a exogenous sequence integrated into the genome of the expression system expresses the fused protein according to claim 19,
preferably, the expression system also comprises RNA,
preferably, the RNA is guide RNA,
preferably, the RNA is sgRNA, and
preferably, a sequence of the sgRNA comprises a sequence that has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a sequence shown as SEQ ID NO: 18 to SEQ ID NO: 65.

25. An expression system, characterized in that the expression system expresses a polynucleotide comprising the polynucleotide according to claim 20, or the exogenous polynucleotide according to claim 20 is integrated into the genome of the expression system.

26. A host cell, characterized by comprising the polynucleotide according to claim 20.

27. A host cell, characterized by comprising the vector according to claim 22.

28. A host cell, characterized by comprising the expression system according to claim 24.

29. A composition, characterized by comprising an effective amount of at least one of the mutant polypeptides according to claim 17, a fused protein comprising the mutant polypeptide according to claim 17, a polynucleotide encoding the mutant polypeptide according to claim 17, or a complementary sequence thereof, a vector comprising a polynucleotide which encodes the mutant polypeptide according to claim 17, or a complementary sequence thereof, or a host cell comprising the polynucleotide which encodes the mutant polypeptide according to claim 17, or a complementary sequence thereof,
preferably, the composition is a kit,
preferably, the composition also comprises RNA,
preferably, the RNA is guide RNA,
preferably, the RNA is sgRNA, and
preferably, a sequence of the sgRNA comprises a sequence that has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a sequence shown as SEQ ID NO: 18 to SEQ ID NO: 65.

30. A base editing system, characterized by comprising the mutant polypeptide according to claim 17, or a fused protein comprising the mutant polypeptide according to claim 17, or a polynucleotide encoding the mutant polypeptide according to claim 17, or a complementary sequence thereof, or a vector comprising a polynucleotide which encodes the mutant polypeptide according to claim 17, or a complementary sequence thereof, or the host cell comprising the polynucleotide which encodes the mutant polypeptide according to claim 17, or a complementary sequence thereof,
preferably, the base editing system also comprises RNA,
preferably, the RNA is guide RNA,
preferably, the RNA is sgRNA, and
preferably, a sequence of the sgRNA comprising a sequence that has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a sequence shown as SEQ ID NO: 18 to SEQ ID NO: 65.

31. A base editing system, characterized by comprising the expression system according to claim 24,
preferably, the base editing system also comprises RNA,
preferably, the RNA is guide RNA,
preferably, the RNA is sgRNA, and
preferably, a sequence of the sgRNA comprising a sequence that has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a sequence shown as SEQ ID NO: 18 to SEQ ID NO: 65.

32. A base editing system, characterized by comprising the host cell according to claim 26,
preferably, the base editing system also comprises RNA,
preferably, the RNA is guide RNA,
preferably, the RNA is sgRNA, and
preferably, a sequence of the sgRNA comprising a sequence that has at least 90% or at least 91% or at least 92% or at least 93% or at least 94% or at least 95% or at least 96% or at least 97% or at least 98% or at least 99% or at least 99.5% or at least 99.8% or at least 99.9% or 100% sequence identity with a sequence shown as SEQ ID NO: 18 to SEQ ID NO: 65.

33. A gene editing method, characterized in that gene editing is performed through the base editing system according to claim 30,
preferably, an editing window of the gene editing covers about 3-10 positions, and
preferably, the editing window of the gene editing covers about 8-10 positions.

34. A method for producing the mutant polypeptide according to claim 17 or the fused protein comprising the mutant polypeptide according to claim 17 by recombination, characterized by comprising the following steps: introducing a vector into host cells to produce transfected or infected host cells, culturing the transfected or infected host cells in vitro, collecting cell cultures, and optionally purifying produced mutant polypeptides or fused proteins; wherein the vector comprising a polynucleotide which encodes the mutant polypeptide according to claim 17, or a complementary sequence thereof.

35. A preparation method of the mutant polypeptide according to claim 17 or a fused protein comprising the mutant polypeptide according to claim 17, characterized by comprising:
(1) adding a polynucleotide encoding the N-terminal fragment of SpRY(D10A) protein, a polynucleotide encoding the TadA8e fragment, and a polynucleotide encoding the C-terminal fragment of SpRY(D10A) protein to a skeleton plasmid to obtain a recombinant expression vector, and
(2) transfecting host cells with the recombinant expression vector to enable the host cells to express the mutant polypeptide or fused protein,
preferably, a nucleotide sequence encoding the N-terminal fragment of SpRY(D10A) protein is shown as SEQ ID NO: 2,
preferably, a nucleotide sequence encoding the TadA8e fragment is shown as SEQ ID NO: 4,
preferably, a nucleotide sequence encoding the C-terminal fragment of SpRY(D10A) protein is shown as SEQ ID NO: 6,
preferably, the skeleton plasmid comprises pCMV or a derived plasmid thereof: ABEmax-SpRY,
preferably, the method comprises removing a TadA dimer from the derived plasmid ABEmax-SpRY, and replacing amino acids located at the 1048th site to the 1063rd site in SpRY(D10A) with TadA8e to construct the recombinant expression vector,
preferably, the vector is a plasmid or virus vector,
preferably, the vector is a plasmid or virus vector used for expression in higher eukaryotes or prokaryotes,
preferably, the eukaryotes are selected from brain neuroma cells and embryonic kidney cells,
preferably, the human embryonic kidney cells comprise HEK293T cells, and
preferably, the brain neuroma cells comprise N2a cells.

36. A treatment method of a genetic disorder, characterized by comprising the following steps: administering a certain amount of at least one of the mutant polypeptide according to claim 17, a fused protein comprising the mutant polypeptide according to claim 17, and a polynucleotide encoding the mutant polypeptide according to claim 17, or a complementary sequence thereof, or any combination thereof that are effective for a genetic disorder to a subject,
preferably, the genetic disorder is phenylketonuria.
